SEQUENCE LISTING (75 sequences)

SEQ ID NO: 1 - protein, 638 aa, Pseudomonas stutzeri
MSDKDSKNTPQVPEKLGLSRRGFLGASAVTGAAVAATALGGAVMTRESWAQAVKESKQKIHVGPGELDDYYGFWSGGHQGEVRVLGVPSMRELMRIPVFNVDSATGWGLTNESRHIMGDSAKFLNGDCHHPHISMTDGKYDGKYLFINDKANSRVARIRLDIMKCDKMITVPNVQAIHGLRLQKVPHTKYVFANAEFIIPHPNDGKVFDLQDENSYTMYNAIDAETMEMAFQVIVDGNLDNTDADYTGRFAAATCYNSEKAFDLGGMMRNERDWVVVFDIHAVEAAVKAGDFITLGDSKTPVLDGRKKDGKDSKFTRYVPVPKNPHGCNTSSDGKYFIAAGKLSPTCSMIAIDKLPDLFAGKLADPRDVIVGEPELGLGPLHTTFDGRGNAYTTLFIDSQVVKWNMEEAVRAYKGEKVNYIKQKLDVHYQPGHLHASLCETNEADGKWLVALSKFSKDRFLPVGPLHPENDQLIDISGDEMKLVHDGPTFAEPHDCIMARRDQIKTKKIWDRNDPFFAPTVEMAKKDGINLDTDNKVIRDGNKVRVYMTSMAPAFGVQEFTVKQGDEVTVTITNIDQIEDVSHGFVVVNHGVSMEISPQQTSSITFVADKPGLHWYYCSWFCHALHMEMVGRMMVEPA

SEQ ID NO: 2 - protein, 636 aa, Pseudomonas aeruginosa
MSDDTKSPHEETHGLNRRGFLGASALTGAAALVGASALGSAVVGREARAAGKGERSKAEVAPGELDEYYGFWSGGHSGEVRVLGVPSMRELMRIPVFNVDSATGWGLTNESKRVLGDSARFLNGDCHHPHISMTDGKYDGKYLFINDKANSRVARIRLDVMKCDRIVTIPNVQAIHGLRLQKVPHTRYVFCNAEFIIPHPNDGSTFDLSGDNAFTLYNAIDAETMEVAWQVIVDGNLDNTDMDYSGRFAASTCYNSEKAVDLGGMMRNERDWVVVFDIPRIEAEIKAKRFVTLGDSKVPVVDGRRKDGKDSPVTRYIPVPKNPHGLNTSPDGKYFIANGKLSPTCTMIAIERLGDLFAGKLADPRDVVVGEPELGLGPLHTTFDGRGNAYTTLFIDSQLVKWNLADAVRAYKGEKVDYIRQKLDVQYQPGHNHATLCETSEADGKWIVVLSKFSKDRFLPTGPLHPENDQLIDISGEEMKLVHDGPTFAEPHDCILARRDQIKTRKIWDRKDPFFAETVKRAEKDGIDLMKDNKVIREGNKVRVYMVSMAPSFGLTEFKVKQGDEVTVTITNLDEIEDVTHGFVMVNHGVCMEISPQQTSSITFVADKPGVHWYYCSWFCHALHMEMCGRMLVEKA

SEQ ID NO: 3 - protein, 653 aa, Pseudomonas versutus
METKQQNGLSRRALLGATAGGAAMAGAFGGRLALGPAVIGAGAAGVATAAGSGAALAASGDGAVAPGQLDDYYGFWSSGQTGELRILGVPSMRELMRVPVFNRCSATGWGITNESILIHERTMSERTRKHLAANGKRIHDNGDLHHVHMSFTEGKYDGRFLFMNDKANTRVARVRCDVMKCDAILEVPNAKAIHGLRPQKWPRSNYVFCNGEDEAPLINDGTTMDDISTYVNVFTAVDADKWEVAWQVLVSGNLDNCDADYEGKWAFSTSYNSEMGMTLPEMTEAEMDHVVVFNIAEIEKAIEAGNFEEINGCKVLDGRKEANSQFTRYIPIANNPHGCNMAPDKKHLVVAGKLSPTVTVLDVTRFDAVFNENADPRSAVVAEPELGLGPLHTAFDGRGNAYTSLFLDSQVVKWNIEEAIRAYAGEQVDPIKDKIDVHYQPGHLKTVMGETLDASNDWMVCLSKFSKDRFLNVGPLKPENDQLIDISGDKMVLVHDGPTFAEPHDAIAVHPSIMSSVIKSVWDRNDPMWAETRKQAEADGVNIDEWTDQIIRDGNKVRVYMSSVAPSFSVESFTVTEGDEVTVIVTNLDEIDDLTHGFTMGNHGVAMEISPQQTSSVTFVAANPGVYWYYCQWFCHALHMEMRGRMMVEPKAA

SEQ ID NO: 4 - protein, 726 aa, Pseudomonas stutzeri
MKPLGFAVVGTSRRWVAGFAILLALMFCVSVAQAKEYEAEQQRIELLFPKVTEISEPEGEYQVRTLADGVGTIYGYAFQSINVTDMPAYSGKPINMQILLDPKGVIVDAYMLEHHEPIVLIGIPEQKVHDFNANYSGIHVDQRVVVGRSSDKSAVTVDAVTGATVTVMVINEIVMRAAHTVAVDLGLVEAGATARPKPALVREDVFQPTSWTELVGNGAIRRMHLTRGQVDDAFKGTEAEGVDVAAAEQRDETFIDLYATHLNPPTIGRNLLGERQYADLMANLKPGEHAFAVLANGEYSFKGSGYVRGGIFDRVQLRQFGDTISFRDLDFIRLSDVYAEGMPEFFEMAIFTAREQYRFDPGSPWNLELLVRRQVGPVESIFTSFEMPYVMPEEYIERVPLTAEELAAIEEANRPLWVNIWYQKSFQVGVILVALALLTVILFLQDKFTQHPNFLKRLRHGYLVFTVVFIGWYALGQLSVVNVLTFVHALVQDFRWELFLTDPVIFILWVFTAASILLWGRGVFCGWLCPFGALQELINEAARKLKIPQYDLPFGVHERLWAIKYIVLLVLFGISLESMMMAEKAAEIEPFKTAITLKFDRQWWFVAYAVFLLVINIFTRKVYCRYVCPLGAGLAITGRFRLFDWLKRRKECGNPCQICANECEVQAIHPDGHINHNECHYCLDCQMTYHNENKCPPLIQKNKRKKRDKKAPVGAELIPVVQVVEP

SEQ ID NO: 5 - DNA, 2181 bp, Pseudomonas stutzeri
  source 1..2181: mol_type = genomic DNA
atgaaacccctaggttttgcagtcgtcggcacctcgcgccgctgggtagctggctttgcgatcctcctggctttgatgttctgtgtatccgtggcacaggcaaaagagtacgaagcagagcagcaacgcatcgagttgcttttcccgaaagtcaccgaaatttccgagcccgagggcgagtaccaggtgcgcacccttgcggacggggtgggcacgatctacgggtacgccttccagagcatcaacgtcacggacatgccggcctactccggcaagccgatcaacatgcagatcctgctcgaccccaagggcgtcatcgtcgatgcttacatgctcgagcaccacgaacccatcgtgctcatcggtattcccgagcagaaggttcacgacttcaacgccaactacagcggcatccacgtcgaccagcgcgtggtagtcggtcgttccagcgacaagagcgcggtcacggtcgacgccgttaccggcgccaccgtgaccgtgatggtgatcaacgagatcgtcatgcgtgcggcccacaccgtggcggtggatctggggctggtcgaggcgggggccaccgcgcgacccaagccggcgctggtgcgcgaggacgtcttccagccgaccagctggaccgagctggtgggcaatggcgccatccgccgcatgcacctgacccgtggccaggtcgacgatgccttcaagggcaccgaggccgagggtgtcgacgttgccgcggcggaacagcgcgacgaaaccttcatcgatctctacgccacgcacctgaacccgccgaccatcggccgcaacctgctgggcgagcgccagtacgccgatctgatggcgaacctcaagccgggcgagcatgccttcgcggtgctggccaacggcgaatactcattcaagggctcgggttacgtgcgtggcggcatcttcgatcgggtgcagctgcgccagttcggcgacaccatcagcttccgcgacctggacttcatccgtctatccgacgtgtatgccgaaggcatgccggaattcttcgagatggcgatcttcactgcccgcgagcagtatcgcttcgatccgggctcgccctggaacctcgagcttctggtgcgtcgccaggtcggcccggtggagagcatcttcaccagcttcgagatgccctacgtgatgcccgaggagtacatcgagcgggtgccgctgaccgccgaagagctggccgccatcgaggaagccaaccggccgctgtgggtcaacatctggtaccagaagagcttccaggtgggggtgatcctggtcgcgctggcgctgctgacggtcatcctcttcctgcaggacaagttcacccagcatcccaacttcctcaagcggctgcgccatggctacctggtcttcaccgtggtgttcatcggctggtatgccctggggcaactgtcggtggtcaacgtgctgaccttcgtccatgcgctggtgcaggacttccgctgggagctgttcctgaccgatccggtgatcttcattctctgggtattcaccgccgctagcattctgctgtgggggcgtggcgtgttctgcggctggctgtgccccttcggcgccctgcaggagctgatcaacgaggccgcgcgcaagctgaagattccccagtacgacctgcccttcggcgttcacgagcggctctgggccatcaagtacatcgtgctgctggtgcttttcggcatctcgctggaatccatgatgatggccgagaaggccgccgagatcgaacccttcaagaccgccatcacgctgaagttcgaccgccagtggtggttcgtcgcctacgcggtgttcctgctggtaatcaacatcttcactcgcaaggtctattgccgctacgtctgcccgctgggcgcggggctggcgatcaccggtcgcttccggctgttcgactggctcaagcggcgcaaggaatgcggcaacccctgtcagatctgtgccaacgaatgcgaagtgcaggcgattcatccggacgggcatatcaaccacaacgaatgccattactgcctggactgccagatgacctaccacaacgaaaacaagtgcccgccgcttattcagaagaacaagcgcaagaagcgcgacaagaaagcgccggtcggggccgagctgatccctgtagtgcaagtggtggaaccctga

SEQ ID NO: 6 - DNA, 2181 bp, synthetic construct
  misc_feature 1..2181: note = Codon optimized NosR gene
  source 1..2181: mol_type = other DNA
atgaagccgttaggtttcgccgtcgtaggcacatcccgccgctgggttgctggtttcgcaatcctgctcgccctgatgttttgtgttagtgtagcgcaggctaaagagtacgaagcagagcaacagcgcattgaattactctttcctaaagtgactgagatctcagaaccggagggcgaataccaagtgcgtacgcttgcagatggtgtgggtacgatctatgggtatgccttccagtccatcaatgtaaccgatatgccggcgtatagtggtaaaccgatcaatatgcagattctcctggacccaaagggggtaatcgtcgacgcttacatgctcgagcaccatgaacctattgtgctgatcggtattccggaacagaaagttcacgacttcaacgctaattacagtgggatccatgttgaccagcgcgttgtcgtaggtcgtagttccgataaatcggcagttaccgtggatgccgtcactggcgctaccgtcacagtaatggtcatcaacgagatcgtcatgcgcgcggctcacacggtcgcagtagatttgggtctcgttgaggctggtgccactgcccgcccgaagccggcattagtgcgcgaagacgttttccagccaacatcgtggaccgagcttgtgggcaatggggctattcgccgtatgcacttaacccgcggtcaagttgacgatgcctttaaaggtactgaagctgagggcgtggatgttgctgccgctgagcagcgcgacgaaacctttattgacttatatgcgacccatcttaacccacctacaatcggtcgtaacttacttggggaacgccagtatgcggatctgatggcaaatctgaaaccaggcgagcacgctttcgccgttttagcgaatggcgagtacagcttcaaagggtccggctatgtgcgcggcgggatttttgatcgcgtgcaactccgtcagtttggggacactatctcttttcgtgatttggattttattcgtctttctgacgtatatgcggaaggtatgccagagtttttcgaaatggcaatttttactgcacgcgaacaataccgctttgacccagggtctccgtggaatctcgaattattggtacgccgtcaagtcggtccagtcgagtcaatctttacttcattcgaaatgccttacgtgatgcctgaagaatacatcgagcgcgtgccgcttaccgctgaagaacttgccgcaatcgaggaagcgaaccgccctctttgggtcaatatctggtatcagaaatcgtttcaagttggggtgatcctggtcgcccttgctctcttaacggtaattttgttccttcaagacaaatttacccagcatcctaacttccttaagcgcttgcgccatggctacctcgtattcaccgtcgtttttattggctggtatgcattaggccagttatctgtcgtgaacgtgcttacattcgttcacgcacttgtgcaggactttcgctgggagctgtttctcaccgaccctgtgatcttcatcctgtgggtcttcaccgctgccagcattcttctctgggggcgcggggtcttttgtgggtggctgtgtccatttggcgccctccaggaacttatcaatgaagccgcgcgcaagctgaagatcccgcagtatgatcttccgtttggcgttcatgagcgcctttgggccattaagtatatcgtactgctggtcttatttggtatctctttagagtcaatgatgatggcggaaaaggccgcagaaattgagccttttaaaactgcgatcacactgaaattcgaccgccaatggtggttcgtggcctatgccgttttcttgttggttatcaatattttcacccgcaaggtttattgccgttatgtttgtcctctcggtgccggcctcgctatcaccggtcgttttcgcttatttgattggcttaaacgtcgtaaagagtgtggcaacccgtgtcagatctgtgctaacgagtgcgaggtacaggcgatccatcctgatgggcacatcaaccacaatgagtgtcactattgcttggactgccaaatgacgtaccataatgaaaacaagtgtccaccgttgattcaaaagaataagcgtaaaaagcgcgacaaaaaagccccggtaggcgcagagttaatcccagtggtccaagtggttgagccttga

SEQ ID NO: 7 - protein, 715 aa, Pseudomonas aeruginosa
MRALRFSAGSWRCVFAWMLLLVGLAAQGAELSELERLRIAQVFPAVERIGDPEGDYGVRRLSKGEETLGYAFQTLSVTDIPAYSGKPINLQVILDPQAVIRDAYVLEHHEPILLIGIPEEKLHAFSARYDGVRADQRVVVGRSSDPQAVTVDAVSGATVTVMVVNEIVMRAAHTVAVSLGLIEDRGNVRPKPAQVRQQPAATANWSELLGNGAIRRLQLSRGQIDDAFKGSEAEGIGEADAAHRDEPFIDLYSALLNPPAVGRSLLGDNQYRELMASLKPGEYAFVVLGDGEYSFKGSGYVRGGIFDRVQLRQFGDIISFRDLDYQRLSDVYAEGMPEFREMAIFVARASQRFDPGSPWTLELLVRRQTGPVAGVFTSFELACQTPEEYLERPQPTAEELAALEEAARPLWLRVWYQKSFQVGVLCTALVLLLAILFLQDRLVRRPRLMQRLRTGYLAFTLVYLGWYSLGQLSVVNVLTFVHALFEGFRWELFLSDPLLFILWTFTAASLLLWGRGVFCGWLCPFGALQELLNELARKLRVPQFQVPFAVHERLWAIKYIILLVLFGLSLESLALAEQAAEVEPFKTAITLGFDRQWWFVAYAVALLVVNLFTRKVYCRYLCPLGAALAIPAKARLFDWLKRRAECGRPCQLCARECEIQAIHPDGRIETNECHYCLDCQMTYHDQDKCPPLVNKRKKRAKSAPADNARIPAENL

SEQ ID NO: 8 - DNA, 2148 bp, Pseudomonas aeruginosa
  source 1..2148: mol_type = genomic DNA
atgcgcgcccttcgtttttccgccggatcgtggcggtgtgtcttcgcctggatgctcctgctggtcggcctcgccgcccagggtgccgaactcagcgagttggagcgcctgcgcatcgcccaggtattccccgcggtcgagcggatcggcgaccccgaaggcgactacggcgtacgccgcctgagcaagggcgaggaaaccctcggctacgccttccagaccctcagcgtcaccgacatcccggcttactccggcaagccgatcaacctgcaggtgatcctcgatccgcaggcggttatccgcgacgcctacgtcctcgaacaccacgaaccgatcctgctgatcggtatcccggaagagaagctgcacgccttcagcgccaggtacgacggcgtgcgcgccgaccagcgggtggtggtcggccgctccagcgacccgcaggcggttaccgtcgacgcggtgagcggcgccacggtgacggtgatggtggtcaacgagatcgtcatgcgcgccgcacatacggtggcggtttccctcggcctgatcgaggaccgcggcaatgtgcggcccaaaccggcgcaggtgcgccagcaaccggcagcgaccgcaaactggagcgaactgctcggcaacggcgcgatccgccgcctgcagttgagtcgcgggcagatcgacgacgccttcaagggcagcgaggccgaaggcatcggcgaagccgacgcggcgcaccgcgacgagccgttcatcgatctctacagcgccctgctcaaccctcccgcggtgggccgcagcctgctcggcgacaaccagtaccgcgaactgatggcgtcgctgaagccaggcgaatacgccttcgtcgtgctcggcgacggcgagtattccttcaagggttccggctacgtgcgcggtggcatcttcgatcgggtccaactgcgccagttcggcgacatcatcagcttccgcgacctcgactaccagcgcctgtcggacgtctatgccgaaggcatgccggagttccgcgagatggcgatcttcgtcgcccgcgccagccagcgtttcgatccgggctcgccctggaccctggagttgctcgtgcggcgccagaccggcccggtggcgggggtattcaccagcttcgagctggcctgccagacgcccgaggaatacctggagcggccgcagccaacggccgaggaactggccgccctggaagaggctgcccggccgctgtggctgcgggtctggtaccagaagagtttccaggtcggggtcctctgtaccgccctcgtcctgctcctggcgatcctcttcctccaggaccgcctggtgcgacggccgcgcctgatgcagcgactgcgcaccggctacctggcgttcaccctggtctacctgggctggtacagcctcggccagctatcggtggtcaacgtgctgaccttcgtccacgcgctgttcgaaggcttccgctgggagctgttcctcagcgacccgctgctgttcatcctctggaccttcaccgcagccagcctgctgctctggggccgcggcgtgttctgcggctggctgtgcccgttcggtgcgctacaggaactgctcaacgaactcgcgcgcaagctccgcgtgccgcagttccaggtgccgttcgccgtgcacgagcggctctgggcgatcaagtacatcatcctgctggtgctcttcggtctctccctggaatccctggcgctggccgagcaggccgcggaggtggagccgttcaagaccgccatcaccctcggcttcgaccgccagtggtggttcgtcgcctacgccgtcgcgctgctggtggtcaacctgttcacccgcaaggtctattgccgctacctctgcccgctgggcgcggccctggcgatcccggccaaggcgcgcctgttcgactggctcaagcgccgtgcggaatgcggcaggccctgccagctctgtgcccgcgaatgcgagatccaggcgatccatcccgacggccgcatcgagaccaacgaatgccactactgcctcgactgccagatgacctaccacgaccaggacaagtgcccgccgctggtgaacaagcgcaagaagcgcgcgaagagcgcgccggcggacaacgcgcggatacccgcggagaacctctga

SEQ ID NO: 9 - DNA, 2148 bp, synthetic construct
  misc_feature 1..2148: note = Codon optimized NosR gene
  source 1..2148: mol_type = other DNA
atgcgcgcacttcgtttctcggcgggctcctggcgctgcgtctttgcatggatgttactgcttgttgggttagctgctcagggtgcagaactctccgaacttgagcgcttacgtatcgcacaagtattccctgctgttgagcgtatcggggatcctgaaggtgattatggggtgcgccgtctctccaaaggggaagaaacattggggtacgcgttccaaacactcagcgtaaccgatattccggcctactccgggaagccaatcaacctccaggttattttggatcctcaggctgtcatccgtgatgcatacgtactcgaacatcacgaaccgatcctcttaattggcatccctgaggaaaagctgcacgccttctctgcccgttacgacggggtacgcgcagaccaacgcgtagtagtggggcgctcaagtgatccacaagctgtaacagtagacgctgtctcgggtgcaacggtgacggttatggtcgtaaatgaaatcgtaatgcgtgcagctcacacagtggccgtgagtcttggcctcattgaggaccgcggcaatgtccgtccaaaaccggctcaggtacgtcagcagcctgcggctacagccaattggtcagagctgttagggaatggcgcaatccgccgcctgcaacttagtcgcgggcagattgatgatgcgttcaaggggagcgaagccgagggtatcggcgaagcagatgccgcgcatcgcgatgaacctttcatcgacttgtacagcgccctgcttaatccgccagcggtaggccgctccctcctgggggacaaccaataccgcgagcttatggcgtctctcaaacctggtgaatacgcttttgtggtgcttggtgatggtgagtactcttttaaagggtctggctacgttcgcggcgggatttttgaccgcgttcagttgcgccaattcggtgatattatttcgtttcgcgacttggattatcaacgcttgtccgacgtgtatgccgaaggtatgccggaattccgcgaaatggcaatctttgtcgcgcgcgcgtcacaacgttttgaccctggttctccgtggaccctggagttattagtacgccgtcaaaccgggccggtggccggcgtgtttacctcctttgagcttgcgtgccaaacaccagaggagtatcttgaacgccctcaacctacagcagaagagcttgcggccctggaagaggctgcgcgcccactttggcttcgcgtttggtaccagaagagttttcaggtcggcgtgctctgcacggcattggtgttactgcttgcgatcttatttcttcaggatcgtcttgtgcgtcgtccgcgcttgatgcaacgtcttcgcacggggtacctggctttcacgttagtttacttaggttggtacagcctcggtcaattgagtgttgtcaatgttcttacttttgttcatgctttgttcgagggttttcgctgggaattgtttctttctgatccgctcctgtttattctttggacgtttacggccgcctcattgttgctgtggggccgcggcgtcttttgtgggtggttatgtccttttggtgctttacaagagttgctcaacgagctggcacgtaagctgcgcgtgccgcaattccaagttccgtttgcggttcatgagcgcttgtgggctattaagtatatcattctgttagttctgttcggcttaagcttagagagcttggcattagctgagcaagctgcagaagtcgaaccatttaagacagcaattaccttagggtttgatcgccagtggtggttcgtggcgtacgccgtcgcattattggtggtcaatctgtttacacgtaaggtctactgccgctatctttgtccgctcggggctgcactggcaattccagccaaagctcgtttgtttgactggctcaaacgtcgcgctgaatgcgggcgcccatgccagttgtgcgcccgcgagtgtgaaatccaagcaattcacccggacggtcgcattgagacgaatgagtgtcactattgcctcgattgtcaaatgacataccatgatcaggataagtgtccgcctcttgtgaacaagcgcaaaaagcgcgccaaatcagctccggcggataacgcacgtatccctgcggagaacctctga

SEQ ID NO: 10 - protein, 729 aa, Pseudomonas versutus
MKILRLVLTIASLLLPALPAAAESVLAQLLPEQNAGELVPGADGFGPIRADLAVAPVLKGGETVAHAFVTSDFVGTTGYSGKPIHTLVALDKDAKVAGVRLVKHSEPIVLIGIPEAKVKALVEGYRGLDLVAEAQSGGTAHEVEIISGATVTVMVIDDSIVRSGLKVARALGLGGLAAETVAAGPKFEIDPDAAPSADWHEMEGDGTLRRLSLDVGQVNAAFAANPDRRAAERALSEAPDTTFIEMQAGLVSVPAIGKALLGDAQAANLQAWLAPGDQAIAVMGRGLYSFKGSGYVRGGIFDRIVLIQDDVSVRFRDRDHRRLNAVAADGAPDFTEMDLFKIPAASGFDPTKPFRIQLLVHREVGPIEKVFHTFDLGYQLPQKYLRSVAAPAPAPEAAAPVAQSDESQAQAQLWKRIWLDSKPKIAGLAAMLLVLTGAFFFQSFATRNERAFYIFRMGFLTVTLIFLGWYANAQLSVVNLMALFGSLVNGFSWQAFLLDPLTFILWFAVAAALLFWGRGAYCGWLCPFGALQELTNQIARKLRIPQWTLPWGLHERLWPVKYMIFLGLFGVSLMSVEQAEHLAEVEPFKTAIILKFIRAWPFVAYAAALLIAGLFVERFYCRYLCPLGAALAIPARMRMFDWLKRYHECGNPCQICAQQCPVQSIHPTGEINPNECINCMHCQVLYQSKTTCPVVIRKLKRREAVAAGSTPKLGQPPAGHPNATRKIEA

SEQ ID NO: 11 - DNA, 2190 bp, Pseudomonas versutus
  source 1..2190: mol_type = genomic DNA
atgaagatcctccgccttgtcctgacgatcgcatcgctgttgctgccggccctgccggccgccgccgaaagcgtgctggcgcagctgctgccggagcagaacgcgggcgagctggtgcccggcgccgacggtttcggaccgatccgcgccgatctggccgtggcgccggtgctgaaggggggcgagaccgtcgcccatgccttcgtcacctcggatttcgtcggcaccaccggctattccggcaagcccatccatacgctcgtcgcgctggacaaggacgcgaaggtggccggcgtccggctggtcaagcattccgaacccatcgtgctgatcggcatccccgaggccaaggtcaaggcgctggtcgagggctaccggggccttgacctcgtggccgaggcgcaatcgggcggcacggcgcatgaggtcgagatcatctcgggcgccacggtcacggtgatggtgatcgacgattccatcgtgcgctcggggctgaaggtcgcgcgcgcgctcggccttggcgggctggccgccgaaaccgtggccgccggcccgaagttcgagatcgaccccgacgcggccccctcggccgactggcacgagatggagggcgacggcaccctgcgccggctgtcgctggacgtgggccaggtgaacgcggcctttgccgccaatcccgaccgccgcgccgccgagcgcgcgctgtccgaggcgccggacaccaccttcatcgagatgcaggccgggctggtctcggtcccggccatcggcaaggcgctgctgggcgatgcgcaggccgcgaacctgcaggcctggctggcgccgggcgaccaggccatcgcggtgatgggccgcgggctctacagcttcaagggctcgggctatgtccgcggcggcatcttcgaccgcatcgtgctgatccaggacgacgtctcggtgcgcttccgcgaccgcgaccatcgccggctgaacgccgtcgctgccgatggcgcgccggatttcaccgagatggacctgttcaagatccccgcggcttcgggcttcgacccgaccaagcccttccgcatccagctgctggtgcatcgcgaggtcgggccgatcgagaaggtcttccacaccttcgacctgggctatcagctgccgcagaaatacctgcgcagcgttgccgcccccgcacccgcgcccgaggcggcggcacccgtggcccagtccgacgaaagccaggcccaggcgcagctgtggaagcggatctggctggattccaagcccaagatcgccgggcttgccgccatgctgctggtgctgacgggggcattcttcttccagagcttcgcgacccggaacgagcgcgccttctacatcttccgcatgggctttctgaccgtgacgctgatcttcctgggctggtatgccaatgcgcagctttcggtcgtgaacctgatggcgctgttcggcagcctggtgaacggcttcagctggcaggccttcctgctggacccgctgaccttcatcctgtggttcgcggtcgccgccgcgctgctgttctggggccggggcgcctattgcggctggctttgccccttcggcgcgctgcaagagctgaccaaccagatcgcgcgcaagctgcgcatcccgcaatggacgctgccctggggcctgcacgagcggctgtggccggtcaaatacatgatcttcctgggccttttcggcgtctcgctgatgagcgtcgagcaggccgagcatctggccgaggtcgagcccttcaagaccgcgatcatcctgaaattcatccgcgcctggcccttcgtggcctatgccgcggcgctgctgatcgccgggctcttcgtcgagcgcttctattgccgctacctgtgcccgcttggcgcggcgctggcgatcccggcgcggatgcgcatgttcgactggctcaagcgctatcacgaatgcggcaatccctgccagatctgcgcccagcaatgcccggtgcagtcgatccacccgacgggcgagatcaatcccaacgaatgcatcaactgcatgcattgccaggtgctttaccagtccaagaccacctgcccggtggtgatccgcaagttgaaacggcgcgaggccgtggccgccggcagcacgcccaagctgggccagcccccggccggccatcccaacgccacccgcaagatcgaagcttga

SEQ ID NO: 12 - DNA, 2190 bp, synthetic construct
  misc_feature 1..2190: note = Codon optimized NosR gene
  source 1..2190: mol_type = other DNA
atgaaaattttgcgtcttgtgttaacaattgcgtcgttgctcctcccggctcttcctgcggcggcggaaagcgtcttagcccaattgttaccagaacaaaatgcaggggaactggtacctggggctgatggcttcgggcctattcgtgcggacctggccgtggccccggtacttaaggggggtgaaaccgttgctcacgcatttgtaacctcggactttgtgggtaccaccggttactcagggaaaccaattcacacgctggtcgctttagacaaagacgctaaagttgcaggggtgcgcctggttaaacattcagagcctattgtcctgattggtattcctgaagccaaagtgaaggccctcgtggaaggttatcgcggcctggacctggttgccgaagctcaatcgggggggacggctcatgaggtagagattatctcaggggcaacagttactgtgatggtgattgacgactcgatcgtccgcagtggtctcaaggtggcacgcgctctcggcctcggggggcttgcggcagaaactgtggccgccggccctaagtttgagatcgacccagatgcagccccgtccgccgattggcacgaaatggagggggatggcacattacgccgcctttcgttagacgtcggccaggttaacgctgccttcgctgcgaacccagatcgtcgcgccgccgagcgtgcattgtcagaagcacctgatacaacattcattgagatgcaggctgggctcgtttcggttccggcgatcggtaaggccctcctcggtgacgcccaagcggcaaatttgcaagcttggttggcccctggtgaccaggccattgcggttatgggccgcggcttatattcgttcaaagggagtggctacgttcgcggtggtatcttcgatcgtatcgtattaatccaagatgacgtgtcagttcgctttcgcgaccgtgatcatcgtcgcctcaacgccgtggccgcggacggtgccccggacttcacagagatggacctgttcaaaatcccggcagcaagtggcttcgacccaacgaaaccgtttcgcattcaacttttagtacatcgcgaagtaggccctattgagaaggtctttcatactttcgacctcggctatcagctgcctcaaaaatatctgcgctcggtcgccgcaccggctccggccccagaggcagccgcacctgttgcccaaagcgatgaatcacaggcgcaagcgcagctctggaagcgcatttggctcgatagtaagccaaaaatcgccggcttggccgcgatgcttttagttctcactggtgcctttttctttcagagtttcgccactcgcaacgaacgcgccttctatattttccgcatgggcttcctcacagtcactctgatttttcttggctggtatgctaatgctcaactgagcgtggtaaatcttatggcactttttgggagtttggtgaacgggttctcgtggcaggcttttctcttggatcctctcacattcattttgtggttcgcagttgccgcagctcttctcttttgggggcgtggtgcttattgtggttggctgtgtccatttggcgcattacaagaattgaccaatcaaattgctcgtaagcttcgcattccgcagtggaccctgccttggggcctgcatgagcgcttatggccggtaaagtatatgatcttccttggtttatttggcgttagtttaatgtcggtggaacaagcagaacatttggccgaagtggaaccttttaaaacggcaattattctgaaatttattcgcgcatggccattcgtcgcttatgcggctgcgctgctcatcgcaggtttgttcgtagaacgcttctactgccgctatctgtgtccgttgggcgcagctctcgcgattccggcccgtatgcgtatgttcgattggctgaagcgttatcacgagtgcggtaatccgtgccagatttgcgcacaacagtgcccggtccagtcgatccacccgaccggggaaattaacccgaacgaatgtattaattgcatgcactgccaagtattataccaatcgaaaaccacttgtccggtagtcatccgcaagctcaagcgccgtgaagcggttgctgcgggttcaacgccgaaattggggcaacctcctgccggtcatccgaatgcgacacgtaaaattgaagcgtaa

SEQ ID NO: 13 - protein, 434 aa, Pseudomonas stutzeri
MRACYTLALTALMIPAGAALAEPQPLTSLPLQAQGENRWTLPAGEYIGQFIIDQPLALHCAPGAVIKAPGQGNVLTVRAANVTIEGCALRDWGRDLTAMNAAIFLEPTATGAQIRNNDLQGPGFGIWADRNRDLLVEGNRIEGDLGLRSQDRGNGIHLFSVRGARVIDNHVWNTRDGIYIDNSNGNSIERNLFEDLRYGVHYMFSHENRVIANVTRRTRTGYALMQSRKLTVIGNRSEHDQNYGILMNYITYSTLKDNFVTDVERGDTGGDSMISGGEGKALFIYNSLFNTIENNHFQRSDLGIHLTAGSEDNRISSNAFVGNAQQVKYVAIRTQEWSVDGRGNYWSDYLGWDRNEDGLGDIAYEPNDNVDRLLWMYPQVRLLMNSPSIEVLRWVQRAFPVIKSPGVQDSHPLMKPPTRGVTEEPMNTTQRPHS

SEQ ID NO: 14 - DNA, 1305 bp, Pseudomonas stutzeri
  source 1..1305: mol_type = genomic DNA
atgcgtgcgtgctacaccctggcgcttaccgcgctcatgattcccgcgggagccgccctggcggaaccgcaaccgctcacctcgctgccgctgcaagcgcagggcgagaaccgctggacactgcccgccggcgaatacatcgggcaattcatcatcgatcaaccgctggcgctgcactgcgcgccgggtgcggtgatcaaggcgccggggcaaggcaacgtcctgaccgtgcgcgcagcgaacgtgaccatcgaggggtgcgcgctgcgtgactgggggcgcgacctcacggcgatgaacgcggcgatcttcctcgaaccgaccgccaccggcgcgcagattcgcaacaacgacctgcaagggccgggcttcggcatctgggccgatcgcaatcgtgacctgctggtcgaaggcaaccgtatcgagggcgacctcggcctgcgttcccaggatcgcggcaacggcatccacctgttctccgtgcgcggtgctcgagtcatcgacaaccatgtctggaacacccgcgacggcatctacatcgacaactccaacggcaacagcatcgagcgcaacctgttcgaggacctgcgctacggcgtgcactacatgttctcccatgagaaccgcgtgatcgccaacgtcacccgccgcacccgcaccggttacgcgctgatgcagagccgcaaactgaccgtgatcggcaaccgctccgagcacgatcagaactacggcatcctgatgaactacatcacctactcgaccctcaaggacaatttcgtcaccgatgtcgagcgtggcgataccggtggcgacagcatgatcagcggcggtgaaggcaaggcgctgttcatctacaactcgctgttcaacaccatcgagaacaaccacttccagcgcagcgatctgggtatccatctgaccgccggctcggaagacaaccgtatttccagcaacgccttcgtcggcaacgcgcagcaggtcaagtacgtcgccatacggacccaggagtggtcggtcgacgggcgcggcaactactggagcgactacctgggctgggaccgcaacgaagacggcctgggcgacatcgcctacgagcccaacgacaacgtcgatcgcctgctgtggatgtatccgcaggtgcgtctgctgatgaacagcccgagcatcgaagtgctgcgctgggtgcagcgcgcgtttccggtgatcaagtcaccgggcgtacaggacagccatccgttgatgaagccacctacccgaggcgtgacagaggaaccgatgaacacaacgcagaggccccactcatga

SEQ ID NO: 15 - DNA, 1305 bp, synthetic construct
  misc_feature 1..1305: note = Codon optimized NosD gene
  source 1..1305: mol_type = other DNA
atgcgcgcttgctacactctcgcccttacggcgttaatgattccggccggggcggctcttgcggaaccacagccgcttacctccttgcctctgcaagcccagggcgaaaatcgctggacgctcccagcaggcgagtacattggccaattcatcattgaccaaccgctggcactccattgtgcacctggggcggttattaaagctccggggcagggtaatgtcttgacagtccgcgcagcaaacgtgacaatcgaggggtgcgcattgcgcgactggggccgcgacctcacggccatgaatgcagccatcttcttggaacctaccgccacgggcgcacagatccgcaataacgatttacaaggcccaggtttcggcatctgggccgaccgtaatcgtgatttgcttgtggaaggcaatcgtatcgaaggtgaccttggcttgcgttcccaggaccgcgggaatggcatccaccttttctctgtacgtggcgcacgcgtaattgacaaccatgtttggaacacacgcgatggcatctatattgacaactccaacggcaattctatcgagcgtaatctgtttgaggacttgcgctacggggtacactatatgttctcacacgaaaaccgcgttatcgcgaacgtcacccgccgcactcgcacaggctacgctttaatgcagagtcgcaaattgaccgttatcggcaatcgctccgaacacgatcaaaactatgggattttaatgaactacattacttatagcaccctcaaagataattttgtcacggatgtcgaacgcggtgatactggcggggattctatgatctcagggggtgaaggtaaagctctctttatctacaatagtctcttcaatacaattgaaaacaaccactttcaacgttccgatctcggcatccatctgacagccggctcagaggataatcgcatctcctccaacgcattcgtggggaatgctcagcaagtcaaatacgtggcaatccgtactcaagagtggagtgtggacggccgcggtaactactggtcggattaccttgggtgggatcgcaacgaagacgggcttggcgatattgcctatgaacctaacgacaatgtagaccgtcttctttggatgtatccacaggttcgcttattgatgaatagcccttcaatcgaggttcttcgttgggttcaacgtgcctttcctgtgatcaaaagccctggtgtccaggattcccatccgcttatgaaacctccaacgcgcggtgtgactgaggaacctatgaacacgactcagcgtcctcatagctaa

SEQ ID NO: 16 - protein, 421 aa, Pseudomonas aeruginosa
MAGRRAGPLLALLLLGLATARAEPVDGLPLRADGDGRWSLAAGRYAGNFVIDRPLHLRCEAGAELDGGGHGSLLTLTSPGITVEGCRLRNWGRNLTELDAAIFVGKAASGAVIRGNDLRGAGFGVWLDATAGAQVLDNRIEGDESVRSQDRGNGIHLYAVKDALVRGNRVSHTRDGVYIDTSNDSSIEANRFEDLRYGVHYMFTHNSRVTDNLTRRTRTGYALMQSRKLTVTGNRSIDDENYGILMNYITYSTLAGNRVEGVRSGSTGDAMISGAEGKALFIYNSLFNRIEGNSFADSALGIHLTAGSEDNRIAGNAFIGNRQQVKYVASREQEWSADGRGNYWSDYLGWDRDDDGLGDVAYEPNDNVDRLIWLYPQVRLLLNSPSIELLRWVQRAFPVVRSPGVRDSHPLMRMPAAEPRP

SEQ ID NO: 17 - DNA, 1266 bp, Pseudomonas aeruginosa
  source 1..1266: mol_type = genomic DNA
atggccgggcgccgggcggggccgctcctcgccctgctcctgctcggcctcgccacggcccgcgcggagccggtcgacggcctgccgctgcgggccgacggcgatggccgctggagcctggcggcgggccgctacgccggtaacttcgtcatcgaccggccgctgcacctacgctgcgaggccggcgccgaactggacggcggcggccacggcagtctgctgaccctgaccagccccgggatcaccgtcgagggctgccggctgcgcaactgggggcgcaacctgaccgaactcgacgccgcgatcttcgtcggcaaggccgccagcggcgccgtgatccgcggcaacgacctgcgcggcgcgggcttcggcgtctggctcgacgccacggcgggcgcgcaggtgctcgacaaccgcatcgagggcgacgaaagcgtgcgctcccaggatcgcggcaacggcatccacctctatgcggtgaaggacgccctggtccgcggcaaccgggtcagccacacccgcgacggggtctacatcgacacctccaacgacagcagcatcgaagccaaccgcttcgaggacctgcgctacggcgtgcactacatgttcacccacaacagccgggtgaccgacaacctgacccggcgcacccgcaccggctacgcgctgatgcagagccgcaagctgaccgtgaccggcaaccgctccatcgacgacgagaactacggcatcctgatgaactacatcacctactcgaccctggccggcaaccgcgtcgagggcgtgcgcagcggcagcaccggcgacgcgatgatttccggcgccgagggcaaggcgctgttcatctacaactcgctgttcaaccgcatcgaaggcaacagcttcgccgacagcgccctgggcatccacctcaccgccggctcggaggacaatcgcatcgccggcaacgccttcataggcaaccgccagcaggtcaagtacgtcgccagccgcgagcaggagtggtccgccgacggccgcggcaactactggagcgactacctgggctgggaccgcgacgacgacggtctcggcgacgtcgcctacgagcccaacgacaacgtcgaccggctgatctggctgtacccgcaggtacgcctgctgttgaacagcccgagcatcgagctgctgcgctgggtccagcgcgccttcccggtggtgcgctcgcccggcgtgcgcgacagccatccgctgatgcggatgcccgccgcggagccgaggccgtga

SEQ ID NO: 18 - DNA, 1266 bp, synthetic construct
  misc_feature 1..1266: note = Codon optimized NosD gene
  source 1..1266: mol_type = other DNA
atggcagggcgccgtgctgggccactgttggccctcttattactcggtctcgcaactgctcgtgctgaaccggtagatggcctcccgttacgtgctgatggcgacggtcgctggtcgctggctgccggtcgttatgccggtaactttgtaattgatcgtcctttgcatttacgttgcgaggctggggccgagcttgatggtggtggtcatgggtcattactcaccctgacgtccccgggtatcacggttgaaggctgccgtctccgcaactggggccgtaatttgacagagcttgatgcagctatttttgttggcaaagctgcttccggcgcagtaatccgcggcaacgacctgcgtggggcgggttttggggtatggttagatgccaccgccggtgcccaggtccttgacaaccgcatcgaaggtgacgagagcgttcgctcccaggaccgcggtaatggtatccatctctacgccgtcaaggacgccctcgtacgcggtaatcgcgtgtctcacacacgcgacggtgtctacatcgacacttccaacgactcctccattgaggcaaatcgctttgaagaccttcgctatggcgtgcactacatgttcacccataattctcgtgtaaccgacaaccttacacgccgcactcgcacaggctatgcattgatgcagagccgtaaactgacagtgacgggtaaccgcagcattgacgacgaaaattacggcatcctcatgaattacatcacatattctacgcttgctggcaatcgcgtggagggggtgcgtagcggctcgacgggtgacgctatgatctcgggtgcggaagggaaggcgcttttcatctacaattctctcttcaaccgtatcgagggcaactctttcgctgattctgcattagggattcacctcaccgccggctcagaggacaatcgtatcgcgggcaacgcctttatcggcaatcgtcaacaggtaaaatacgtcgcttcgcgcgaacaggagtggtctgccgatggccgcggtaactactggtctgactacctcggctgggatcgcgatgatgatggcttgggtgacgtcgcatacgaaccaaacgacaatgtggatcgtttaatttggttgtacccacaagtccgtttacttctgaatagcccttccattgagttacttcgctgggtgcaacgtgctttcccggtggtacgcagtccaggtgtgcgcgattcccaccctttgatgcgcatgccagccgcggagcctcgcccttaa

SEQ ID NO: 19 - protein, 443 aa, Pseudomonas versutus
MRSLLTLALALLLAWPALAAEHVVAPGQGSLAQAIAGAAPGDVLRLQDGVHAGPVAIDRPLTVTGSRAAVVDGQGRGTVVTIAAPDVTLQGFSVTGSGMANKDLDAGVKILKGADRAQVRQLRLTGNMHGIDVHGGRDAQVVGNEIIGTRDPRMNERGNGIYVWNSPGTLVQGNSVRYGRDGIFSNASGDSAYRDNLFRDLRFAVHFMYTRNTEVSGNVSIGNHLGFAIMFSDRAVIRDNRSLGDREHGLMLNYANNADVTGNLIRGGTKKCLFIYNAHKNLIWDNRFQDCGIGIHFTAGSERNVLTANAFVGNREQVKYVGTRHIEWSHEGRGNFWSDHPGFDLNGDGIADGVYRPNDLMDHILWSQPAAALLTGAPAVQLIRWSQQSFPATLPGGVQDSAPLMRPLTIPVPPEIAAYEAEVAGRWAKGTYDDIDPDDLTSH

SEQ ID NO: 20 - DNA, 1332 bp, Pseudomonas versutus
  source 1..1332: mol_type = genomic DNA
atgcgcagcctgctgacccttgcccttgccctgctgcttgcctggcccgcgctcgcggccgagcatgtcgtggcgccgggccagggcagccttgcccaagccatcgccggggccgcccccggcgatgtgctgcgactgcaggacggggtccatgccggccccgtcgccatcgaccgtcccttgaccgtcaccggcagccgtgccgcggtggtggacgggcagggacggggcacggtcgtcaccatcgccgcgcccgacgtcaccttgcagggcttttccgtcacgggctcgggcatggcgaacaaggatctggacgccggggtcaagatcctcaagggcgccgaccgggcgcaggtgcggcagttgcggctgaccgggaacatgcacggcatcgacgtgcatggcggccgcgacgcccaggtcgtcggcaacgagatcatcggcacccgcgacccgcgcatgaacgagcgcggcaacggcatctatgtctggaacagccccggcacgctggtgcagggcaattccgtccgctacggccgcgacggcatcttttcgaacgccagcggcgacagcgcctatcgcgacaacctgtttcgcgacctgcgctttgccgtgcatttcatgtatacccgcaacaccgaggtgtcgggcaatgtcagcatcggcaaccacctgggcttcgccatcatgttctcggaccgggcggtgatccgcgacaaccgcagcctgggcgaccgcgagcatgggctgatgctgaactatgccaacaatgccgacgtgaccggcaacctgatccgcggcggcaccaagaaatgcctgttcatctataacgcccacaagaacctgatctgggacaaccgcttccaggattgcggcatcggcatccacttcaccgccgggtccgagcgcaacgtgctgaccgcaaacgcctttgtcggcaatcgcgagcaggtgaaatacgtgggcacccgccatatcgaatggagccacgaggggcgcggcaatttctggtccgaccatccgggcttcgacctgaacggcgacggcatcgccgacggcgtctatcgccccaacgacctgatggaccatatcctgtggtcgcagcccgccgccgcgcttctgaccggcgcccccgccgtgcagctgatccgctggagccagcagagctttcccgccaccctgccgggcggcgtgcaggacagcgcgcccctgatgcgacccctgaccatccccgtcccgcccgagatcgcggcctacgaggccgaggtcgcggggcgttgggcaaaaggaacctacgatgacatcgaccctgacgatctcacgtctcactaa

SEQ ID NO: 21 - DNA, 1332 bp, synthetic construct
  misc_feature 1..1332: note = Codon optimized NosD gene
  source 1..1332: mol_type = other DNA
atgcgtagtcttttgacacttgcgctggcattgctcttagcttggccggcattggcggcagagcacgttgttgccccgggtcagggttccttagcccaggctatcgcgggtgctgctccaggcgatgtgcttcgcttgcaagacggggtacatgcggggccagtagctattgatcgtccgttgactgtcacgggcagccgcgctgcggtggttgacggtcaaggccgtggtacggttgtaactatcgccgcaccggatgttacactgcaagggttctcggtaacggggtctgggatggctaataaggaccttgatgccggggtgaaaattttaaagggggcagatcgtgcgcaggtgcgccaattacgcttgacggggaacatgcatggtatcgacgtccatggggggcgcgacgcgcaagtcgttggtaacgaaattatcggtactcgcgacccacgcatgaatgagcgcggtaacggtatctatgtatggaatagcccgggcactttagttcaagggaatagcgttcgctacgggcgcgatggcatctttagcaacgcgtcgggcgattctgcataccgcgataatttgttccgtgacctgcgtttcgctgttcactttatgtatacacgcaatactgaggtttctggtaacgtctccatcgggaatcatcttgggttcgcgattatgttctccgaccgcgctgttattcgcgacaatcgctctctgggtgatcgtgagcatggcctcatgcttaattacgcgaataacgccgatgtcaccggcaatttaatccgcggtggtactaagaaatgcctcttcatctataacgcacacaagaacttgatctgggacaatcgtttccaagactgtgggatcggcatccactttacggccggttccgaacgcaatgttttaacggctaacgcgtttgtaggtaatcgtgagcaggtaaagtacgtcgggacacgtcacattgagtggtctcacgaaggtcgcggtaacttctggtctgatcatcctgggtttgacctcaatggtgatggtattgcagatggtgtctaccgcccaaatgatcttatggaccatatcctttggagtcaacctgctgcagcacttcttaccggggcacctgctgtacagttgatccgttggtcacagcaatcattcccagcgacgttgccggggggtgtgcaagactccgcaccacttatgcgcccgctcactatcccagtcccaccagagatcgcggcgtatgaagcagaggtagcgggtcgttgggcaaagggcacgtatgacgacattgacccagacgatttaactagtcattga

SEQ ID NO: 22 - protein, 308 aa, Pseudomonas stutzeri
MNAVEIQGVSQRYGNMTVLHDLNLNLGEGEVLGLFGHNGAGKTTSMKLILGLLQASEGRVQVLGRDPRTTDVRRHLGYLPENVTFYPQLTGRETLRHFARLKSTPLGQVDDLLEQVGLAHAADRRVKTYSKGMRQRLGLAQAVLGEPRLLLLDEPTVGLDPIATQELYLLIDRLRQTGTSVILCSHVLPGVEAHINRAAILAKGRLQAIGSLKQLRSEAGLPVRIRASGISQSEAWLERWANAGHSAQRLGEDGVEVIAINGHKLPLLRELLGESEPDDVEIHQPSLEDLYRYYMERAGDVQAAEGRV

SEQ ID NO: 23 - DNA, 927 bp, Pseudomonas stutzeri
  source 1..927: mol_type = genomic DNA
atgaatgccgtcgagatccagggcgtcagccagcgttacggcaacatgaccgtgttgcatgatttgaacctgaacctcggcgaaggcgaggtactggggctgttcggccataacggcgccggcaagaccaccagcatgaaactgattctcggcctgctgcaggccagcgaagggcgcgtccaggtactgggtcgcgatccgcgcaccaccgacgtgcgccgtcacctgggctatctgccggagaacgtcaccttctatccgcaactgacggggcgcgagaccctgcgtcatttcgcgcgcctgaagagcacgccgctgggccaggtggacgatctgctcgagcaggtcggcctcgcccacgccgccgatcgccgcgtgaagacctattccaagggcatgcgccagcggctcggcctggcccaggcggtgctcggcgagccgcgcctgctgctgctcgacgaacccaccgtggggctcgaccccatcgccacccaggagctctacctgctgatcgatcggctgcgccagacgggtaccagcgtgatcctctgttcccacgtgctgcccggcgtggaggcgcatatcaaccgtgcggcgattctcgccaaggggcgcctgcaggccatcggcagcctcaagcagctgcgcagcgaggccggcctgccggtgcgtatccgcgccagtggcatcagtcagagcgaggcctggctggagcgctgggccaatgcggggcactcggcgcagcgtctcggcgaggatggcgtcgaggtgatcgccatcaacggccacaagctgccgttgctgcgtgagctgctcggggaaagcgaacccgacgatgtggaaatccaccagccttcgctggaggatctgtaccgctattacatggagcgcgccggcgatgtgcaggccgcggagggcagggtatga

SEQ ID NO: 24 - DNA, 927 bp, synthetic construct
  misc_feature 1..927: note = Codon optimized NosD gene
  source 1..927: mol_type = other DNA
atgaacgctgtggaaattcaaggggtttctcagcgctatgggaatatgactgtactgcatgatttgaacttgaacttaggggaaggtgaagtgcttgggttattcggtcacaatggcgctgggaagacaacatcaatgaaacttatcctgggcctcctgcaagcatccgagggtcgtgtccaggtgctgggccgtgacccacgtaccactgatgtgcgtcgccacctcggttacttacctgaaaatgtcacgttttatcctcaacttacagggcgcgagactttacgtcacttcgcccgtctgaagtccacccctctgggccaagtggacgatctgctggagcaagttggtctcgctcacgctgcagaccgccgcgtgaaaacctactcaaaaggtatgcgccaacgcttgggcttagcgcaggcggtactgggtgagcctcgccttctgctgctggatgaaccaactgttgggttggaccctattgcgacgcaagagctctatctcttaattgatcgcctccgtcaaactggtacgtcggttatcctgtgttcacacgttcttccaggtgtagaggctcacattaatcgcgcagcgatccttgctaagggtcgcctccaagccatcgggtcactcaaacaattacgcagcgaagccgggttacctgtccgtattcgtgcctcgggtatttcccagagtgaggcctggttagagcgttgggcgaatgcggggcattcggcgcaacgtttaggcgaggatggcgttgaagtgattgcgatcaatgggcacaaactgccattgctccgtgagttattgggggaatcagaaccagacgacgtggaaattcaccaaccgtcgctggaagatctgtatcgctactatatggaacgtgcaggtgacgtgcaggccgcagaagggcgcgtataa

SEQ ID NO: 25 - protein, 304 aa, Pseudomonas aeruginosa
MSLVEIDGATLRYGALTALSGLDLRLEPGEVLGLLGHNGAGKTTTIKLVLGLLAPSEGRVRVLGHDARSLEARRQLGYLPENVTFYPQLSGAETLRHFARLKGVAPAEAARLLEQVGLGHAARRRLKTYSKGMRQRLGLAQALLGEPRLLLLDEPTVGLDPLATVELYQLLDRLRGQGTGIVLCSHVLPGVETHIDRAAILAGGRLQVAGSLAELRRKAALPTRVRLASPHNPQWLERWHRAGLAARRLDDQRIEVLLDDAERDGVLEALLAAREFDLEILPPSLEDLYRHHMSPAPAGATPCP

SEQ ID NO: 26 - DNA, 915 bp, Pseudomonas aeruginosa
  source 1..915: mol_type = genomic DNA
gtgagcctggtcgagatcgacggcgcgaccctgcgctacggcgccctcaccgccctgagcgggctcgacctgcgcctggagcccggcgaggtgctcggcctgctcggccacaacggcgccggcaagaccaccaccatcaagctggtcctcggcctgctggcccccagcgaaggccgcgtgcgggtcctcggccacgatgcgaggagcctggaggcgcgccgccagctcggctacctgccggagaacgtgaccttctacccgcagctcagcggcgcggaaaccctgcgccacttcgcccgcctcaagggcgtggcgccggccgaagccgcgcgcctgctggaacaggtcggcctcggccatgccgccaggcggcgcctgaaaacctactcgaagggcatgcgccagcgcctcggcctggcccaggcgctgctcggcgaaccgcgcctgctgctgctcgacgaaccgacggtgggcctcgacccgctggccaccgtcgagctctaccaattgctcgaccgcctgcgcggccagggcaccgggatcgtcctttgctcccatgtgctgcccggcgtcgagacgcacatcgaccgcgccgcgattctcgccggcggccgcctgcaagtggccggcagcctcgccgaattgcgccgcaaggcggctctgccgacccgcgtgcgcctggccagcccgcataacccgcagtggctcgaacgctggcaccgggccggcctggcggcgcggagactggacgaccagcgcatcgaggtactgctggacgatgccgagcgcgacggcgtgctggaagcgctgctggccgcgcgcgagttcgacctggaaatcctgccgccgtcgctggaggacctctatcgccaccacatgtcccccgcccccgccggagccacgccatgcccgtag

SEQ ID NO: 27 - DNA, 915 bp, synthetic construct
  misc_feature 1..915: note = Codon optimized NosF gene
  source 1..915: mol_type = other DNA
atgagccttgtggaaattgacggtgcgactttacgctacggggcgctcactgcgctttctggcttggatttacgcttagagccaggggaagtcttaggcttattagggcataacggcgctggcaagactacaacgatcaagcttgttctcgggttgctcgcgccgagcgaaggccgcgtccgtgtcctcgggcatgatgcgcgcagtttggaagctcgccgccagttaggttatctgccagagaacgtgaccttttatcctcaattaagtggggcggagactttacgccatttcgctcgtttaaagggtgtagcccctgccgaggctgcacgtcttctggagcaagtcggtttaggtcacgcggcgcgccgccgtcttaaaacgtactctaaagggatgcgccagcgcttgggcctcgcacaggccctgcttggggagccgcgccttcttttgttagatgaaccgacggtaggtttggacccacttgcgaccgtggagttatatcaactcttggatcgccttcgtgggcaggggaccgggatcgttttatgtagtcatgttctgccaggggttgagactcatatcgatcgcgctgctattctggcaggcggccgtcttcaggtcgcaggttcgcttgcagaattacgccgtaaagctgctttgccaacccgcgttcgtctcgcttcaccgcacaatcctcaatggttggaacgctggcaccgtgctggtctggcagcccgccgcttagacgaccaacgcatcgaggtattattagacgatgcggagcgcgatggtgtacttgaggctctgctggcggcccgtgaatttgacctggaaatcctgccaccgagcttagaagatctgtatcgccaccacatgtccccagcgccggcgggtgcaacgccatgtccatga

SEQ ID NO: 28 - protein, 299 aa, Pseudomonas versutus
MTSTLTISRLTKRFQTVEALAEVSLDLGPGMRVALLGHNGAGKSTMMKIILGLIPFDAGEVRVCGAAPGSAQARMQVAYLPENAAFHPALTGEEQIRHYLSLRGESPRRAMELLERVGLAKAARRRIGTYSKGMRQRVGLAQTLIGRPRLLVLDEPTSGLDPVSRRDFYALLDGLAAEGAAILLSSHVLTEVEARTDRILILSQGRLVAEGTLAELRRRAALPVGLTVVPAPGAAEALAAALPQARLAGDGTLHLACAQDEKLGLLARIAGLGGQVADLDVIPPSLEDIYSHFSRRDGQ

SEQ ID NO: 29 - DNA, 900 bp, Pseudomonas versutus
  source 1..900: mol_type = genomic DNA
atgacatcgaccctgacgatctcacgtctcactaagcgcttccagaccgtcgaggcgctggccgaggtctcgctggacctggggccggggatgcgcgtcgcgcttttgggccataacggcgcgggcaagtccacgatgatgaagatcatcctgggcctgatcccctttgacgcgggcgaggtccgggtctgcggcgccgcgcccggctcggcccaggcgcggatgcaggtggcctatctgccggaaaacgccgccttccacccggccctgaccggagaggagcagatccgccactacctttccctgcgcggcgaaagcccgcgccgggccatggaactgctggagcgggtggggctggccaaggccgcgcgccgccgcatcggcacctattccaagggcatgcgccagcgcgtcggcctggcccagacgctgatcgggcggccgcggcttctggttctggacgagccgacctcggggctcgacccggtgtcgcggcgcgacttctatgcgctgctcgacgggctggcggccgagggggcggcgatcctcttgtcctcgcatgtgctgaccgaggtcgaggcccgcaccgaccgcatcctgatcctgtcgcagggccggctggtggccgagggcacgctggccgagttgcgccgccgcgccgccctgccggtggggctgaccgtggtccccgcgcccggcgcggccgaggcgctggccgccgcgttgccgcaggcgcggctggcgggcgacggcacgctgcacctggcctgcgcgcaggacgaaaagctgggcctgctggcgcgcatcgccgggcttggcggccaggtcgcggacctggacgtgatcccgcccagcctcgaggacatctacagccatttcagccggagggacgggcaatga

SEQ ID NO: 30 - DNA, 900 bp, synthetic construct
  misc_feature 1..900: note = Codon optimized NosF gene
  source 1..900: mol_type = other DNA
atgacaagtacactgacgattagccgcttaactaaacgtttccagaccgtagaggcattagctgaagttagtctcgacctcggcccgggcatgcgcgtagcgcttttgggccataatggtgcgggtaaatcaacaatgatgaaaatcattttggggctcatcccgtttgacgctggcgaggtccgcgtctgcggggccgccccagggtcagctcaagcccgcatgcaagtagcatacttaccagagaacgctgctttccaccctgcacttactggtgaagagcaaattcgccattatttatcactccgcggggagtctccacgtcgtgctatggaattgctggagcgcgttgggttagctaaggccgcacgccgtcgtatcggtacgtattcaaagggtatgcgtcaacgtgtaggcttggcccaaacgcttatcggtcgcccgcgcctcctcgtcttagatgagccaacctcaggtctggacccggtgagccgccgcgacttttatgcgttactcgacggcctcgctgctgagggtgctgccattctcctctccagccatgttctcacagaggtcgaagcgcgtaccgaccgtattttaatcctgagccaaggccgccttgtggccgagggtacgttagcagagttgcgccgtcgcgcagctttgccagttggccttacggtcgtaccagcgccaggtgctgctgaagcccttgcagcagcgctgcctcaagcccgtctggcaggtgatgggacgctgcatctcgcatgtgcacaagatgagaaattggggttactggctcgtatcgccgggttagggggtcaagtcgccgatctggacgtaatcccgccttctctcgaagatatttattcacactttagtcgccgcgacgggcagtaa

SEQ ID NO: 31 - protein, 275 aa, Pseudomonas stutzeri
MNQVWNIARKELSDGLRNRWLLAISLLFAVLAVGIAWLGAAASGQVGFTSIPATIASLASLATFLMPLIALLLAYDAIVGEDEGGTLMLLLTYPLGRGEILLGKFVGHGLILALAVLIGFGGAALAIALLVEDIELGLLLWAFGRFMVSSTLLGWVFLALAYVLSGKVNEKSSAAGLALGIWFLFVLVFDLVLLALLVLSEGKFSPELLPWLLLLNPTDIYRLINLSGFEGGGAMGVLSLGSDLPVPNAVLWLCLLAWAGASLLLAYGIFRRRLA

SEQ ID NO: 32 - DNA, 828 bp, Pseudomonas stutzeri
  source 1..828: mol_type = genomic DNA
atgaaccaggtctggaacatcgcccgcaaggaactcagcgatggcctgcgcaaccgctggctgctggccatcagcctgttgttcgcggtgctggccgtggggatcgcctggctcggtgcggccgcctcggggcaggtggggttcacctcgatcccggcgaccatcgccagcctggccagcctggccaccttcctgatgccgctgatcgcgctgctgctggcctatgacgccatcgtcggcgaggacgaaggcggcacgctgatgctgctgctgacctacccgctggggcgcggcgagatcctgctcggcaagttcgtcggccacgggctgatccttgccctggcggtgttgatcggcttcggcggcgccgctctggccatcgccctgctggtcgaggatatcgagctgggcctgctgctctgggccttcggtcgcttcatggtttcctccacgctgctgggctgggtgttccttgccctggcctacgtgctcagcggcaaggtcaacgagaaatccagcgccgccggcctggcgctggggatctggttcctgttcgtgctggtgttcgacctggtgctgctggcgctgctggtgctcagcgaaggcaagttcagccccgagttgctgccctggctgctgctgctcaaccccaccgacatctaccggctgatcaacctgtccggcttcgagggcggcggcgccatgggcgtgctgtcgctgggcagcgacctgcccgtgccgaacgccgtgctctggctctgcctgttggcctgggcaggcgcatcgctgctgctggcctatggcatcttccgtcggcgcctggcctga

SEQ ID NO: 33 - DNA, 828 bp, synthetic construct
  misc_feature 1..828: note = Codon optimized NosY gene
  source 1..828: mol_type = other DNA
atgaaccaagtgtggaacattgcgcgtaaagagttgtctgatggtctccgtaaccgctggttacttgctatttcattactctttgccgtattggctgtcggcatcgcttggctcggcgccgctgcgtctggtcaagttggtttcacgtctatcccagcgactatcgcgtccttagcgtcactggcaactttcctgatgcctttaattgcacttcttctcgcttatgatgccattgtcggtgaagatgagggggggactctcatgttattactgacctacccgttgggccgtggggaaatcttactgggcaagttcgtggggcatggcttaatcttggctttagctgtactcatcgggtttggcggggcggctttagctattgccctcttggtcgaggacattgaacttgggctgctgctctgggcattcggccgcttcatggtgagtagtacgctcctcgggtgggtatttttggccctcgcttacgttctttccggcaaagtcaatgagaagtcttctgcggcaggtctggcgctggggatttggttcttgtttgtacttgtgttcgatcttgtccttttagctctgctggtattgtccgaagggaagtttagtcctgaattattgccttggcttttgttattgaatcctaccgacatctatcgtcttattaatttgtccggctttgaaggtggtggggcgatgggggtattgtcattggggagtgaccttcctgtaccaaatgcggtcttatggctttgcctcctggcttgggccggcgcgtcattgcttttggcgtatggcattttccgtcgtcgcttagcttga

SEQ ID NO: 34 - protein, 275 aa, Pseudomonas aeruginosa
MPVVWTIARKELADGLRNRWLLAISLLFALLSVGIAWFGAAAAGQVGFTSVPATVASLTSLATFLMPLIALLLAYDAIVGEEEGGTLLLLLTYPLGRGQLLLGKFLGHGLILALATLIGFGSAALAILALVPEVEAAILLGAFGRFMGSSLLLGCVFLALAYALSSRASEKSSAAGQALGLWFFFVLLFDLALLAILVLSQGHLSPRLLPWLLLLNPTDLYRLINLSGFDAAAAGGVVPLASDLPVSASALWLALALWACAALALAHGLFRRRPI

SEQ ID NO: 35 - DNA, 828 bp, Pseudomonas aeruginosa
  source 1..828: mol_type = genomic DNA
atgcccgtagtctggaccatcgcccgcaaggaactggccgacggcctgcgcaatcgctggctgctggcgatcagcctgctgttcgccctgctctcggtgggcatcgcctggttcggcgccgccgcggccggccaggtcggcttcacttcggtgccggcgacggtcgccagcctgaccagcctggccaccttcctgatgccgctgatcgccctgctgctggcctacgacgccatcgtcggcgaggaggaaggcggcaccctgctgctgctcctgacctacccgctggggcgcggccagttgctgctcggcaagttcctcggtcacggcctgatcctcgccctggccaccctgatcggcttcggcagcgcggcgctggcgatcctcgcgctggtgccggaggtcgaggcggccatcctgctgggcgccttcggccgcttcatgggttcctcgctgctgctcggctgcgtgttcctcgccctggcctacgccctgagcagccgcgccagcgagaaatccagcgccgccgggcaggcgctcggcctgtggttcttcttcgtcctgctgttcgacctggccctgctggcgatcctggtcctcagccagggtcacctgagcccgcggctgctgccctggctgttgctgctcaacccgaccgacctctaccggctgatcaacctgtccggcttcgacgcagccgccgccggcggggtggttcccctggccagcgacctgccggtgtccgccagcgccctctggctggccctggccctctgggcctgcgctgccctggcgctggcccacggcctgttccgccgccgccccatctga

SEQ ID NO: 36 - DNA, 828 bp, synthetic construct
  misc_feature 1..828: note = Codon optimized NosY gene
  source 1..828: mol_type = other DNA
atgccggtcgtgtggacgattgcacgtaaagagctcgccgacggccttcgtaatcgctggctccttgcaatctcgctgttgttcgctctcttatccgtagggattgcctggttcggcgcagccgcagctggccaagttgggttcacttcggttcctgcgacagttgctagtctgacatccctggccactttcctcatgcctttgatcgctttgctcttggcgtacgatgctatcgtaggggaagaggaaggtggcacactcctgctcctgcttacttacccattaggccgcggccagctcctcctcgggaagtttctcggtcatggcttgatcctcgcgctggcaactctgattggtttcggttccgcagcacttgctatccttgcactcgtgcctgaagtggaagccgcaattttactcggtgcattcggtcgctttatggggtcctcacttcttttaggttgcgtgttcctggctttggcttatgccttatcgagccgtgctagcgaaaagtcttctgctgcagggcaagccttagggttgtggtttttcttcgtgttgcttttcgacttagcattattggctattctggttctgagccaaggtcacttatcaccacgtctccttccatggttactcttgcttaacccgacagacctgtaccgtctgattaacttaagcggctttgatgcagccgccgccgggggggtcgtccctttggcttccgacttgccagtatcagccagtgcgctttggctcgcgttggcgttatgggcttgcgcggcgttagcccttgctcatggcttgttccgccgtcgcccaatttaa

SEQ ID NO: 37 - protein, 274 aa, Pseudomonas versutus
MIRRILSTAALEFRIALRNRWVAIATALMVVFALVLAAAGAAPTGDIGADRLSVVVASLTSLAVYLVPLLALLMSFDAVAGEVERGTLPLLLTYPVARAEVLAGKLVAHMAILALAVGAGYGAAALAAVWSDPASTAGLPALWRLMWSSTLLGATFLGAGYALSSIARRPSGAAGLAVGLWLGLVVLYDLALLALIVADGGGGFTTEVLPVALLANPADAFRLFNLSAAQATAAAAGVGGAAATIPLWQSALSVLAWPLAALGLAIAAFRKVTP

SEQ ID NO: 38 - DNA, 825 bp, Pseudomonas versutus
  source 1..825: mol_type = genomic DNA
atgatccgccgcatcctttccaccgcggcgctggagttccgcatcgcgctgcgcaaccgctgggtcgccatcgccaccgcgctgatggtggtctttgcgctggtgctggcggcggcgggtgccgcgcccaccggcgacatcggcgccgaccggctgtcggtcgtcgtcgcctcgctgacctcgcttgccgtctacctggtgccgctgctggcgctgctgatgagcttcgatgccgtcgccggcgaggtcgagcgcggcaccttgcccttgctgctgacctatcccgtggcgcgggccgaggtgctggccggcaagctggtcgcgcatatggcgattctggcgctggcggtgggcgcgggctacggcgcggcggccctggcggcggtctggagcgatccggcctcgaccgcggggcttccggcgctgtggcggctgatgtggagctcgaccctgctgggcgcgaccttcctgggcgccggctatgcgctgtccagcatcgcgcgccggccctcgggcgcggccgggctggcggtcgggctgtggctggggctggtggtgctttacgacctggccctgctggcgctgatcgtcgccgatggcggcggcggcttcaccaccgaggtgctgccggtcgcgctgcttgccaacccggccgacgccttccgcctgttcaacctctcggccgcccaggccaccgccgccgctgccggcgtcggcggggccgccgcgaccatcccgctttggcaatcggcgctgtcggtcctggcctggccgctggccgcgcttggccttgccattgccgcattccgaaaggtcacgccatga

SEQ ID NO: 39 - DNA, 825 bp, synthetic construct
  misc_feature 1..825: note = Codon optimized NosY gene
  source 1..825: mol_type = other DNA
atgatccgtcgcattctcagtacagcggccttggaatttcgtattgctctccgcaaccgttgggtggccattgcgactgcactcatggtagtgtttgctttagttcttgcggctgctggcgcggcgccaacgggtgatattggcgcggaccgtttgagcgtagttgtggcctcgctcacttcactcgcagtgtatcttgttcctctgttagcacttcttatgtcgttcgacgctgtagctggcgaggtcgagcgcgggacgcttccgttgttgcttacctatccggttgcacgcgcggaggtactcgcggggaagctcgttgcccacatggcaattttagctttagccgtgggtgctggttatggtgcagcagcgctcgcagcagtttggtcggaccctgccagcactgcagggcttccagcactttggcgtcttatgtggagttccacattgctgggtgcaacctttctgggcgcaggctacgcactttcctcgattgcacgccgcccttctggcgcagccgggttagcggtggggctgtggttgggcctcgttgttctttatgatctcgcattgttggcactcatcgtagccgatggtggtggtggtttcaccacggaagtgctcccagtggctttattagcgaacccagccgatgcgtttcgcctttttaatttgtccgctgcacaagcaacagctgccgctgcaggggttggtggcgctgcagccactatcccactttggcaaagcgccctcagcgttctggcgtggccattggctgcattgggtcttgcgattgcggcgttccgtaaggttactccgtaa

SEQ ID NO: 40 - protein, 341 aa, Pseudomonas stutzeri
MGRASLQPVIAVALAAALTGCLFQDKVEDFGGPTMGSTYSVKYVASDGAAAKAQLQRETESILAQLDEQLSTYRADSDIEAFNALPAGQCMAMPESARELVLAGQQLSQESDGALDLTIQPLLNLWGFGPQGRREQVPSAEEIARARETVGHRHLQVVGEQLCKDAAVEVDFNSIAAGYAVDLVAQRLEALGVESYLVEITGELKARGRKPGDAPWRIAIEAPRDNERVAQRVIELDGYGVSTSGDYRNYFERDGKRYSHTLDPQTGAPIEHRLAAVTVVDPSALRADGLSTVLMVLGPERGLAYAAEHRIAALFVIHEEQEFISKSTAAFDELFGAGAEQ

SEQ ID NO: 41 - DNA, 1026 bp, Pseudomonas stutzeri
  source 1..1026: mol_type = genomic DNA
atgggccgagcgtcactccaacccgtcatcgctgtcgccctggcggcagccctgacgggttgtctgtttcaggacaaggtagaagacttcggtggcccgaccatgggcagtacctactcggtgaagtacgttgccagtgatggtgcggctgccaaggcacaactgcagcgcgagaccgaatcgattctggcccagctggacgagcagttgtcgacctaccgcgccgattcggatatcgaagccttcaatgccctcccggcagggcagtgcatggccatgcccgagtccgcccgcgagctcgtgctggctgggcagcagctgtcgcaggaaagcgacggcgcactggacctgaccattcagccgctgctcaacctctgggggttcggcccgcaggggcgccgtgagcaggtaccttctgccgaggaaattgccagggcccgcgagactgtcggtcaccggcatctgcaggttgtcggcgagcagctgtgcaaggatgcggccgtcgaggtcgatttcaacagcatcgctgcgggttacgctgtcgatctggtggcgcaaaggctggaagcgctcggggtcgaaagctatctggtggaaatcactggcgagctcaaggcacgaggccgaaagccgggtgacgcgccgtggcggatcgccatcgaagcgccgcgggataatgagcgtgtggcgcagcgcgtcatcgagctcgatggctatggcgtttccacctcgggtgactaccgcaattatttcgagcgtgatggcaagcgctactcgcatacgctggatccgcaaaccggtgccccgatcgagcatcgcctggcggccgtgacggtcgtcgatccatcagcactgcgcgcggacgggttgtctaccgtcctgatggtgctggggccggaacgtggcctggcgtatgccgcggagcacaggatcgcagcgctcttcgtgattcatgaagagcaggaattcatcagcaaaagcaccgcagccttcgatgagctgttcggtgcgggagcagagcaatga

SEQ ID NO: 42 - DNA, 1026 bp, synthetic construct
  misc_feature 1..1026: note = Codon optimized apbE gene
  source 1..1026: mol_type = other DNA
atggggcgtgcgagcctgcaaccagtaattgctgttgctttagcggcggcgctgaccggttgccttttccaggataaggtcgaagattttggggggccgaccatgggtagtacttactctgtcaaatacgtcgccagcgatggcgcggctgcgaaagctcaactccaacgcgaaactgagagtatcttggcacagttggatgagcaactctccacttatcgtgctgacagcgatattgaagcctttaacgccctgccagctggtcaatgcatggcgatgccagaaagcgctcgcgaactggtattagcaggtcagcaacttagtcaagagtccgacggtgcgctggatctcacaatccaaccattactgaatttatggggtttcggccctcagggccgccgtgaacaagtcccatcggcagaggaaatcgctcgtgcccgtgagaccgtgggtcatcgtcatctccaggttgtgggcgaacaactgtgcaaggacgcagctgtcgaggtggattttaactcaattgcggcaggctatgctgtggacctcgttgcacagcgtcttgaagcgctcggtgttgagtcttatctggtggaaatcactggggaactgaaggcccgtggtcgcaaaccaggcgatgcgccgtggcgtatcgcgatcgaagctccgcgtgataacgagcgtgtggcgcagcgtgtaattgaattggatggttacggcgtcagcacctcaggtgactatcgcaactattttgaacgtgacgggaagcgttactctcacactctcgatcctcagaccggcgcacctatcgaacaccgcctggctgcggtaacggtggtagatccatcagctcttcgcgcagatgggttgtccactgtgcttatggtgctcggcccagaacgtgggttggcgtatgcggctgagcaccgtatcgcggcactctttgtgatccatgaggaacaggaatttatcagcaaatccactgccgcttttgacgagctcttcggggcgggggcagagcagtga

SEQ ID NO: 43 - protein, 333 aa, Pseudomonas aeruginosa
MAGLAALALVLAGCGDTLESFGGPTMGSTYSIKYVRGASAPDVQTAKAAVEAILAEVDRQMSTYRDDSLVSRFNALPAQSCMELPPPMLELLRYGGELSEQSQGAFDMTVEPLMNLWGFGPQARVEKVPSAEQIAAVRRDVGHRHLRIDGQRLCKDAAVQLDFDSIAAGYTVDAVGERLKELGVRSYLAEITGELKAEGRKPDGTPWRIAIEAPREGQRVAQQVLALDGYGVSTSGDYRNYFEENGQRYSHTFDPRTGAPIDHHLASVTVIDPSTRNADGLSTLLMALGPEEGYRFAEKHRLAALFVSRQGNGFDSRTTPRYEQLFGNQGERP

SEQ ID NO: 44 - DNA, 1002 bp, Pseudomonas aeruginosa
  source 1..1002: mol_type = genomic DNA
atggccggactggcagcgctggcgctggtcctggccggttgcggcgacaccctggaaagcttcggcggcccgaccatgggcagcacctattcgatcaagtacgtacgcggcgcctcggcgcccgacgtccagacggcgaaagccgcggtggaggcgatcctcgccgaggtcgaccggcagatgtccacctaccgcgacgattccctggtctcgcgtttcaatgcgctgccggcgcagtcctgcatggagctgccgccaccgatgctggagttgctgcgctacggcggcgagttgtcggagcagagccagggcgccttcgacatgaccgtcgagccgctgatgaacctctggggcttcggtccgcaggcgcgggtagagaaggttcccagcgccgagcagatcgccgcggtgcgccgtgacgtcggccatcgacacctgcgcatcgacggccagcgcctgtgcaaggacgccgccgtacaattggacttcgacagcatcgccgctggctatacggtggacgcggtaggcgaacgcctgaaggagctcggcgtgcgcagctacctggcggaaatcaccggcgagttgaaagccgaagggcgcaagccggacggcacgccctggcgcatcgctatcgaagcgccgcgcgagggccagcgggtcgcccagcaggtactggcgctggacggctacggcgtctcgacctcgggcgattaccgcaactatttcgaagagaacggccagcgctattcgcacaccttcgacccgcgcaccggcgcgccgatcgaccatcacctggcttcggtcacggtgatcgatccgtcgacgcgcaacgccgacggcctgtcgaccctgctgatggcgctcggtccggaggagggctaccgtttcgccgagaagcaccgactggcggcgctgttcgtcagtcgccagggcaacggtttcgacagccgcaccacgccacgctacgagcaactgttcggcaaccaaggagaacgtccatga

SEQ ID NO: 45 - DNA, 1002 bp, synthetic construct
  misc_feature 1..1002: note = Codon optimized apbE gene
  source 1..1002: mol_type = other DNA
atggcggggttagcagcgctggcacttgttctggccgggtgtggtgacactttggaaagtttcggcggtcctactatggggtccacttattccattaagtacgttcgcggcgcttcggctcctgacgtacagaccgccaaggccgcagttgaggccattttggcggaagtggaccgccagatgagtacataccgtgacgacagcctggtttctcgttttaatgcattgccagcgcaatcgtgtatggaattgccgccacctatgctcgaattgttacgctacggtggtgagctttcagaacaaagtcagggcgcttttgatatgactgtcgagcctctgatgaacctgtgggggttcggcccgcaagcccgcgtggaaaaggttccttcggcagaacagatcgccgcggtgcgccgcgatgtggggcatcgtcatctccgtatcgacggtcagcgcttgtgtaaagatgcggccgtacaactcgactttgattcaattgcagccggctacacagtagatgcagtgggcgagcgtttaaaggagcttggtgtccgttcatatttggccgaaattactggcgaattgaaagcagaggggcgcaagcctgatgggacgccatggcgtatcgctattgaggcgccacgcgagggccagcgtgtggcccagcaggtgcttgcgctcgatggctacggtgtgtctacgtctggggattaccgcaactattttgaggaaaacggtcaacgctatagtcacacgtttgaccctcgtacaggtgcgccgatcgatcaccacttagcgtcagtcacagtgatcgacccgagcactcgcaacgcagatggcttatccactctcctgatggcgctcggtcctgaagagggttaccgttttgcagagaaacaccgtcttgcggcgctttttgtgagccgccaagggaatggtttcgactcacggacaactccgcgctatgaacaactgttcgggaatcagggtgaacgcccataa

SEQ ID NO: 46 - protein, 302 aa, Pseudomonas versutus
MSRRRFLTITAAVALAPAALRAQPGRHWVGQALGARASIRIDHPEAEAITARCLAEIDRLEGILSLYRPDSALSRLNRDAALEAPPFELLECLSLAGTVHRASGGLFDPTVQPLWSLWAEAALAGRRPTADERRAALARTGWERVRLDAARITLEPGMALTLNGIGQGYVADRVAALLEAEGLGDILIDTGELRALGSRPDGTDWPVRLAEGGAVGLRGRALATSAPLGTSFDQAGRDGHILDPRSGAPARPAWRAISISAPGAGLADALSTAACLAGDRQEILALLARFDGARLEAAPRGA

SEQ ID NO: 47 - DNA, 909 bp, Pseudomonas versutus
  source 1..909: mol_type = genomic DNA
ctgtcccgccgtcgctttctgaccatcaccgccgccgtggcgcttgcgcccgcggccctgcgtgcccagcccggccggcattgggtcgggcaggcgcttggcgcccgcgcctcgatccgcatcgaccaccccgaggccgaggcgatcaccgcccgctgcctggccgagatcgaccggctggaaggcatcctcagcctctatcgtcccgacagcgccctgtcgcggctgaaccgggacgcggcgctggaggcgccgcccttcgagctgctggaatgcctgtcgctggccggcacggtgcatcgcgccagcggcgggctgttcgaccctaccgtgcagccgctctggtcgctctgggccgaggccgccttggccggccgccgccccaccgcggacgagcgtcgcgcggcgctggcgcgcaccggctgggagcgggtgcggctggacgcggcgcgcatcacgctggagccggggatggcgctgacgctgaacggcatcggccagggctatgtcgccgaccgcgtcgccgcgcttctcgaggccgaggggctgggcgacatcctgatcgacaccggcgaattgcgcgccctgggcagccggcccgacggcaccgactggccggtgcggctggccgagggcggcgcggtgggcctgcgcggccgggcgctggcgacatcggcgccgctgggcaccagcttcgaccaggcggggcgcgacggccatatcctcgacccgcgcagcggcgcgccggcgcgtccggcctggcgcgcgatcagcatctcggcccccggcgcgggcttggccgatgcgctgtccaccgccgcctgcctggccggcgaccggcaggagatcctggcgctgctggcccgtttcgacggcgcccggcttgaagcggcgccgcgcggggcatag

SEQ ID NO: 48 - DNA, 909 bp, synthetic construct
  misc_feature 1..909: note = Codon optimized apbE gene
  source 1..909: mol_type = other DNA
atgtcacgccgtcgtttcctgactattactgcagctgtggcgctggctcctgctgcgctgcgcgcgcaaccaggccgccattgggtaggccaagcattaggtgctcgtgcatccattcgcattgaccatccagaggctgaggctattacagcacgctgcttggctgagattgatcgcctggagggtatcctttccctttatcgtccggacagcgcgcttagtcgcttgaaccgcgatgccgcgctggaagcgccaccattcgaactcctggaatgcttaagtctcgctggcacggtccatcgtgcgtctggtggtctctttgacccaactgtgcagcctctttggtcattatgggccgaagccgcgcttgccggtcgccgtccaactgcggatgaacgtcgtgcagctttagcacgtacaggttgggagcgcgtacgcttggacgcagctcgtatcaccttagagccaggcatggctctcaccctcaatggtattgggcaaggttatgtcgccgaccgcgttgctgccctgctcgaagcagagggccttggtgacatcctgattgacaccggggaactccgcgctctcgggagtcgcccggacgggaccgattggccagtacgcctcgccgaaggtggggccgtggggcttcgtgggcgtgcattagctacctccgcgccgctgggtacctccttcgaccaggctgggcgcgacggccatatcttggacccacgttcgggcgcgccagctcgcccggcttggcgtgcgatctcaatttcggcccctggcgcaggtcttgcagacgccttaagcaccgccgcatgtttggctggtgaccgtcaggaaattttagcgttacttgcacgcttcgacggggcccgccttgaggcggctcctcgcggtgcctga

SEQ ID NO: 49 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcactgtccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 50 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcagcatccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 51 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcatgttccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 52 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcattctccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 53 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcacattccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 54 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcaaaatccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 55 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcaatgtccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 56 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcacagtccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 57 - DNA, 1917 bp, Pseudomonas stutzeri
  source 1..1917: mol_type = genomic DNA
atgagcgacaaagattccaagaacactccgcaagtgcccgagaaactcggcctgagccgccgcggcttcctcggcgccagcgcagtcaccggtgccgccgttgctgccacggctctcggcggcgcggtgatgacccgcgaatcctgggcgcaggccgtcaaggagtccaagcagaagatccacgtcggccccggcgagctggatgactactacggcttctggtccggcggtcaccagggtgaagtccgcgtgctgggcgtgccgtcgatgcgcgagctgatgcgtatcccggtgttcaacgtcgactcggccaccggctgggggctgaccaacgaaagccgccacatcatgggcgacagcgccaagttcctcaacggtgactgccaccacccgcacatctccatgaccgacggcaagtacgatggcaagtacctgttcatcaacgacaaggccaacagccgcgttgcgcgtatccgtctggacatcatgaagtgcgacaagatgatcaccgtgccgaacgtgcaggcgatccacggtctgcgtctgcagaaggtgccgcacaccaagtacgtattcgccaacgccgagttcatcatcccgcacccgaacgatggcaaggtcttcgatctgcaggacgagaacagctacaccatgtacaacgccatcgatgcggaaaccatggaaatggccttccaggtcatcgttgacggcaacctcgacaacaccgacgccgactacactggccgtttcgctgctgctacctgctacaactcggagaaggccttcgatctgggcggcatgatgcgtaacgagcgcgactgggtggtggtgttcgatatccacgccgtcgaagcagcggtcaaagctggcgatttcatcaccctgggcgactccaagacgcctgtgctcgatggtcgcaagaaggatggcaaggacagcaagttcacccgttacgtgccagtgccgaaaaacccgcacggctgcaacacctcctccgatggcaaatacttcatcgccgccggcaagctctcgccaacctgctcgatgatcgccatcgacaagctgcccgacctgttcgccggcaagctggccgatccgcgtgatgtgatcgtgggtgagcctgagctgggtctcggcccgctgcacaccaccttcgacggccgcggtaacgcctacaccacgctgttcatcgacagccaggtggtcaagtggaacatggaagaagctgttcgtgcctacaagggcgagaaggtcaactacatcaagcagaagcttgatgtgcactaccagccgggtcacctgcacgcgtcgctgtgtgaaaccaatgaagccgatggcaagtggctggtagcatcgtccaagttctccaaggaccgcttcctgccggttggcccgctgcatcccgagaacgaccaactgatcgacatctccggcgacgagatgaagctggtacatgacggcccgacctttgccgaaccgcatgactgcatcatggctcgccgtgatcagatcaagaccaagaagatctgggaccgcaacgatccgttcttcgccccgaccgtggaaatggcgaagaaggacggcatcaacctcgataccgacaacaaggtcattcgcgacggcaacaaggtgcgcgtgtacatgacctcgatggcgccggcgttcggcgtgcaggagttcaccgtcaagcagggcgatgaagtcaccgtgaccatcaccaacatcgaccagatcgaagacgtctcccacggcttcgtggtggtcaaccatggcgtgagcatggagatcagcccgcagcagacttcttccatcacctttgtcgctgacaagccaggcctgcactggtactactgcagctggttctgccatgcgctgcacatggaaatggtcggccgcatgatggtcgagccggcctaa

SEQ ID NO: 58 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgctcagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 59 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtggcaagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 60 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgtgtagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 61 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgttcagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 62 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgcatagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 63 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgaaaagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 64 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgatgagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 65 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgcagagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 66 - DNA, 1911 bp, Pseudomonas aeruginosa
  source 1..1911: mol_type = genomic DNA
atgagcgacgacacgaaaagcccccacgaagaaacccacggcctgaaccgccgcggcttcctcggcgcctcggcgctgaccggagccgccgccctggttggcgccagcgccctgggcagcgcggtggtcggccgcgaggcccgggccgcgggcaagggcgagcgcagcaaggccgaggtcgcccccggcgaactggatgagtactacgggttctggagcggcggacattccggcgaagtacgcgtgctcggcgtgccgtcgatgcgcgagctgatgcgcataccggtgttcaacgtcgactcggccaccggctggggcctgaccaacgagagcaagcgggtcctcggcgacagcgcgcgcttcctcaacggcgactgccaccatccgcacatctcgatgaccgacggcaagtacgacggcaagtacctgttcatcaacgacaaggccaacagccgggtcgcgcgcatccgcctggacgtcatgaaatgcgaccgcatcgtcaccattcccaacgtccaggcgatccacggcctgcgcctgcaaaaggtgccgcatacccgctacgtgttctgcaacgccgagttcatcatcccccatcccaacgacggctcgaccttcgacctgtccggcgacaacgccttcaccctgtacaacgccatcgacgccgagaccatggaagtggcctggcaggtgatcgtcgacggcaacctcgacaacaccgacatggactacagcggcaggttcgccgcctccacctgctacaactcggaaaaggccgtcgacctcggcggcatgatgcgcaacgagcgcgactgggtggtggtgttcgacatcccgcgcatcgaggccgagatcaaggcgaagcgcttcgtcaccctcggcgactcgaaggtgccggtggtcgacggccggcgcaaggacggcaaggacagcccggtgacccgctacatcccggtaccgaagaacccccacgggctgaacacctcgccggacggcaagtacttcatcgccaacggcaagctctcgccgacctgcaccatgatcgccatcgagcgcctcggcgacctgttcgccggcaagctggccgacccgcgcgacgtggtggtgggcgagccggaactgggcctcggcccgctgcacaccactttcgatggccgaggcaacgcctataccacgctgttcatcgacagccagttggtgaagtggaacctcgccgacgcggtgcgcgcctacaagggcgagaaggtcgactacatccgccagaagctcgacgtgcagtaccagccggggcacaaccacgccactctgtgcgagaccagcgaagccgacggcaagtggatcgtggtgtcgagcaagttctccaaggaccgcttcctgcccaccggtccgctgcacccggagaacgaccagttgatcgacatttccggcgaggaaatgaagctggtccacgacggcccgaccttcgccgaaccgcacgattgcatcctcgcccgccgcgaccagatcaagacccgcaagatctgggaccgcaaggacccgttcttcgccgagacggtcaagcgcgcggaaaaggacggcatcgacctgatgaaggacaacaaggtcatccgcgagggcaacaaggtccgcgtctacatggtctcgatggcgccctccttcggcctcaccgagttcaaggtgaagcagggcgacgaagtcaccgtgaccatcaccaacctcgacgagatcgaggacgtgacccacggcttcgtcatggtcaaccacggcgtctgcatggagatcagcccgcaacagacctcgtcgatcaccttcgtcgccgataagcccggggtgcactggtactactgcagctggttctgccacgccctgcacatggaaatgtgcgggcggatgctggtggaaaaggcttga

SEQ ID NO: 67 - DNA, 1962 bp, Pseudomonas versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgcctgtccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 68 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgcgcatccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 69 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgctgttccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 70 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgcttctccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 71 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgccattccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 72 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgcaaatccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 73 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgcatgtccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 74 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgccagtccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

SEQ ID NO: 75 - DNA, 1962 bp, Paracoccus versutus
  source 1..1962: mol_type = genomic DNA
atggaaaccaaacagcaaaacggcctgagccgccgcgcgcttctgggcgcgaccgccggcggtgcggccatggccggcgcgttcggggggcgcctggcgctgggaccggccgtgatcggtgcgggcgccgcgggcgtcgccaccgccgccggcagcggcgcggcgctggccgccagcggcgacggcgcggtcgcgccgggccagctggacgactattacggcttctggtcctcgggccagaccggcgagctgcgcatcctgggcgttccctcgatgcgcgagctgatgcgggtgccggtcttcaaccgctgctcggccaccggctggggcatcaccaacgagtcgatcctcatccacgaacgcaccatgagcgagcggacgaggaagcaccttgccgccaacggcaagcgcatccacgacaacggcgacctgcaccacgtccacatgtcctttaccgagggcaagtatgacggccgcttcctgttcatgaacgacaaggccaatacccgcgtggcgcgggtgcgctgcgacgtgatgaaatgcgacgccatcctggaggtgccgaacgccaaggccatccacggcctgcgcccgcagaaatggccgcgcagcaactatgtgttctgcaacggcgaggacgaggcgccgctgatcaacgacggcaccacgatggacgacatctcgacctatgtgaacgtcttcaccgccgtcgatgccgacaagtgggaagtcgcctggcaggtgctggtctcgggcaacctcgacaactgcgatgccgattacgagggcaaatgggccttctcgacctcgtataactcggagatgggcatgaccctgcccgagatgaccgaagccgagatggaccatgtcgtggtcttcaacatcgccgagatcgaaaaggccatcgaggccggcaattttgaagagatcaacggctgcaaggtgctggacggccggaaagaggcgaacagccagttcacccgctacatcccgatcgccaacaacccgcatggctgcaacatggccccggacaagaagcacctggtcgtcgcgggcaagctgtcgcccacggtgacggtgctggacgtgaccaggttcgacgcggtgttcaacgagaatgccgatccgcgcagcgccgtggtggccgagccggaactgggcctgggcccgctgcacaccgccttcgacgggcgcggcaacgcctatacctcgctgtttctcgacagccaggtggtcaagtggaacatcgaggaggcgatccgcgcctatgccggcgagcaggtcgatccgatcaaggacaagatcgacgtgcattaccagcccggccacctcaagacggtgatgggcgagacgctggacgccagcaacgactggatggtctgctcgtccaagttctcgaaggaccgcttcctgaacgtcgggccgctgaagccggaaaacgaccagctgatcgacatctcgggcgacaagatggtgctggtccatgacggcccgacctttgccgagccgcatgacgccatcgccgtgcatccctcgatcatgtcgagcgtcatcaagtcggtctgggaccgcaacgatcccatgtgggccgaaacccgcaagcaggccgaggccgacggcgtcaacatcgacgaatggaccgaccagatcatccgcgacggcaacaaggtgcgggtctacatgtccagcgtcgcgcccagcttctcggtcgaaagcttcaccgtgaccgagggcgacgaggtcacggtcatcgtcaccaaccttgacgagatcgacgacctgacccacggtttcaccatgggcaaccacggtgtcgccatggagatctcgccccagcagacctcgtcggtgactttcgtcgccgccaatccgggggtgtactggtattattgccagtggttctgccatgcgctgcacatggaaatgcgcggccgcatgatggtcgagccgaaggcggcctga

(19) United States
(12) Patent Application Publication
(10) Pub. No.: US 2023/0383266 A1
(43) Pub. Date: Nov. 30, 2023

(54) VARIANT OF NITROUS OXIDE REDUCTASE PROTEIN AND METHOD OF REDUCING CONCENTRATION OF NITROUS OXIDE IN SAMPLE

(71) Applicants: Samsung Electronics Co., Ltd., Suwon-si, Gyeonggi-do (KR); Konkuk University Industrial Cooperation Corp, Seoul (KR)

(72) Inventors: Woo Yong Shim, Suwon-si, Gyeonggi-do (KR); Seung Hoon Song, Suwon-si, Gyeonggi-do (KR); Anurag Kumar, Seoul (KR); Jung-Kul Lee, Seoul (KR); Jae-Young Kim, Suwon-si, Gyeonggi-do (KR); Yu Kyung Jung, Suwon-si, Gyeonggi-do (KR)

(21) Appl. No.: 18/318,262

(22) Filed: May 16, 2023

(30) Foreign Application Priority Data
May 24, 2022 (KR) 10-2022-0063723

(51) Int. Cl. C12N 9/06; C12N 15/70

(52) U.S. Cl. CPC C12N 9/0044; C12Y 107/02004; C12N 15/70

(57) ABSTRACT

Provided are polynucleotides encoding variants of nitrous oxide reductase proteins, recombinant microorganisms including a foreign gene encoding a variant of a nitrous oxide reductase protein, and a composition comprising the recombinant microorganism or the variant of a nitrous oxide reductase protein for use in removing nitrous oxide in a sample.

### CROSS-REFERENCE TO RELATED APPLICATION

This application is based on and claims priority under 35 U.S.C. § 119, and all of the benefits accruing therefrom, to Korean Patent Application No. 10-2022-0063723, filed on May 24, 2022, in the Korean Intellectual Property Office, the content of which is incorporated by reference herein in its entirety.

### BACKGROUND

### 1. Field

The disclosure relates to recombinant microorganisms including foreign genes that encode variants of nitrous oxide reductase proteins, compositions for reducing a concentration of nitrous oxide in a sample that include the recombinant microorganisms or the variants of nitrous oxide reductase proteins, and methods of reducing a concentration of nitrous oxide in a sample by using the recombinant microorganisms or the nitrous oxide reductase proteins.

### 2. Description of the Related Art

Nitrogen oxide (NOx) is one of the air pollutants mainly discharged during combustion of fuels, and examples of various nitrogen oxides (also referred to as NOx) include N₂O, NO, N₂O₃, NO₂, N₂O₄, N₂O₅, and the like. The main air pollutants among these examples are NO and NO₂. N₂O, along with carbon dioxide (CO₂), methane (CH₄), and freon gas (e.g., chlorofluorocarbons (CFCs)), absorbs and stores heat in the atmosphere and thus is a main cause of the greenhouse effect, and is one of the six greenhouse gases regulated under the Kyoto Protocol. N₂O has a Global Warming Potential (GWP) of 310, exhibiting a higher warming effect per unit mass than carbon dioxide having a GWP of 1 and methane having a GWP of 21. In addition, nitrogen oxide is also a cause of smog and acid rain, forms second-generation ultrafine dust through chemical reactions in the air, and adversely affects respiratory health by increasing the ground-level ozone concentrations.

Most nitrogen oxide removal processes are chemical reduction methods, such as a selective catalytic reduction (SCR) method and a selective non-catalytic reduction (SNCR) method, and techniques using scrubbing, adsorption, and the like are being applied. Chemical methods have drawbacks in terms of the cost of energy and catalyst required throughout the whole process, the treatment of secondary waste generated during the process, and the like. In addition, in the case of an SCR or SNCR method, N₂O, which is another greenhouse gas, may be generated as a result of incomplete reduction during reducing NO and NO₂. Unlike chemical techniques having such problems, biological processes are environmentally friendly and have advantages such as a relatively simple principle, no use of extreme conditions (including e.g., high temperature and high pressure), and low generation of secondary waste or wastewater. In such biological processes, a microorganism serving as a biological catalyst may be used instead of a chemical catalyst, to oxidize or reduce NOx or to fix NOx as part of a cell.

However, despite advances, alternative methods of biological denitrification are still required.

### SUMMARY

Denitrification microorganisms reduce nitrogen oxide to N₂ through a dissimilatory reductive process. In several previous studies, many denitrification microorganisms such as Pseudomonas putida, Pseudomonas denitrificans, Pseudomonas stutzeri, Paracoccus denitrificans, Klebsiella pneumonia, and the like have been reported.

Provided in an aspect are recombinant microorganisms including foreign genes encoding variants of nitrous oxide reductase proteins.

Provided in an aspect are compositions for use in reducing a concentration of nitrous oxide in a sample comprising the recombinant microorganisms or the variants of the nitrous oxide reductase proteins.

Provided are methods of reducing a concentration of nitrous oxide in a sample that includes nitrous oxide, the method including contacting the sample with the variant of the nitrous oxide reductase protein or with a recombinant microorganism to reduce a concentration of nitrous oxide in the sample.

Provided in an aspect are variants of nitrous oxide reductase proteins and polynucleotides encoding the variants.

Additional aspects will be set forth in part in the description which follows and, in part, will be apparent from the description, or may be learned by practice of the presented embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram of a vector introduced into E. coll.

FIG. 2 is a diagram showing the results of converting N₂O to N₂ using a recombinant E. coli including a wild-type nosZ gene and a variant of the nosZ gene.

FIG. 3 is a diagram showing results of converting NO in the form of Fe(II)EDTA-¹⁵NO to N₂ using a recombinant E. coli including a wild-type nosZ gene and a variant of the nosZ gene.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. “Or” means “and/or.” “At least one” is not to be construed as limiting “a” or “an.” As used herein, the term “and/or” includes any and all combinations of one or more of the associated listed items. As used herein, the term “or” and “and/or” includes any and all combinations of one or more of the associated listed items. Expressions such as “at least one of,” when preceding a list of elements, modify the entire list of elements and do not modify the individual elements of the list.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, ±10% or ±5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The expression “increase in expression” or “increased expression” as used herein refers to a detectable increase in expression of a given gene. In particular, the expression “increased expression” refers to a level of gene expression of a given gene in a genetically modified cell (for example, a genetically engineered cell) that is greater compared to an expression level in a comparative cell of the same type as a cell without the same genetic modification (for example, a native cell or a “wild-type” cell). For example, the expression level of a gene in the modified cell may be increased by about 5% or more, about 10% or more, about 15% or more, about 20% or more, about 30% or more, about 50% or more, about 60% or more, about 70% or more, or about 100% or more, than the expression level of the same gene in a non-engineered cell of the same type, for example, a wild-type cell or a parent cell of the modified cell. Cells with increased expressions of proteins or enzymes may be identified using any method known in the art.

The increase in expression of a gene may be achieved by increasing the copy number of the gene. The expression “copy number increase” as it relates to a gene refers to an increase in copy number due to introduction or amplification of a given gene, and may also include a case in which a gene not present in a non-engineered cell is obtained by genetic engineering. The gene introduction may be achieved through a vehicle such as a vector. The introduction may include transient introduction in which a gene is not integrated into the genome, or insertion of a gene into the genome. The introduction may be achieved by, for example, introducing a vector into which a polynucleotide encoding a desired polypeptide is inserted into a cell, and then replicating the vector in a cell or integrating the polynucleotide into the genome. The expression “copy number increase” may refer to an increase in the number of copies of a gene encoding one or more polypeptides of a complex when several polypeptides form the complex and exhibit activity of nitrous oxide reductase as a complex.

The introduction of the gene into the cell may be performed by known methods such as transformation, transfection, electroporation, and the like. Here, the gene may be introduced through a vehicle, or may be introduced as it is. The term “vehicle” as used herein refers to a nucleic acid molecule capable of delivering another nucleic acid linked to the vehicle. In terms of a nucleic acid sequence that mediates the introduction of a particular gene, the vehicle as used herein is understood to be used interchangeably with a vector, a nucleic acid structure, and a cassette. A vector may include, for example, a plasmid-derived vector or a virus-derived vector. A plasmid may include a circular double-stranded DNA loop to which additional DNA may be ligated. The vector may include, for example, a plasmid expression vector or a viral expression vector including, for example, replication defective retrovirus, adenovirus, and adeno-associated virus, or a combination thereof.

The gene engineering described herein may be performed by a suitable molecular biological method known in the art.

The term “parent cell” as used herein refers to an original cell, for example, a cell of the same type as the engineered cell, but without the genetic modification. Regarding a particular genetic modification, the “parent cell” refers to a cell that does not have such a particular genetic modification, but may be the same as a genetically engineered cell for other circumstances. Thus, the parent cell may be used as a starting material to produce a genetically engineered microorganism having an increased (or decreased) expression level of a gene encoding a given protein (for example, a protein having a sequence identity of about at least 75% with respect to a nitrous oxide reductase). The same comparison may be applied to other genetic modifications.

The term “gene” as used herein refers to a nucleic acid fragment encoding the information for expressing a particular protein, and may or may not include a regulatory sequence with a 5′-noncoding sequence and/or a 3′-noncoding sequence, or may be free of a regulatory sequence.

A polypeptide is a polymer chain comprised of amino acid residue monomers which are joined together through amid bonds (peptide bonds). In general, a polypeptide may include at least 10, 20, 50, 100, 200, 500, or more amino acid residue monomers.

The term “sequence identity” of a nucleic acid or polypeptide as used herein refers to the degree of identicalness of nucleotide (bases) or polynucleotide sequences or amino acid residues of a polypeptide between sequences, and is obtained after aligning both sequences to be as identical as possible (best match) in a specific comparison region. The sequence identity is a value measured by optimally aligning and comparing two sequences in a specific comparison region, and a portion of the sequences in the comparison region may be added or deleted compared to a reference sequence. A sequence identity percentage may be, for example, calculated by steps of: comparing two optimally aligned sequences throughout the comparison region; determining the number of positions in both sequences where identical amino acids or nucleotides appear to obtain the number of matched positions; dividing the number of the matched positions by the total number of positions within the comparison range (i.e., a range size); and multiplying the result by 100 to obtain a sequence identity percentage. The sequence identity percentage may be determined by using a known sequence comparison program, and examples of the program are BLASTn™ (NCBI), BLASTp™ (NCBI), CLC Main Workbench (CLC bio), MegAlign™ (DNASTAR Inc), and the like.

The term “genetic modification” as used herein refers to an artificial alteration in a composition or a structure of a genetic material in a cell.

The term “corresponding” as used herein refers to an amino acid position of a polypeptide or protein of interest that aligns with a stated position of a standard polypeptide or protein (e.g., position L452 of SEQ ID NO: 1), when the amino acid sequence (e.g., SEQ ID NO: 1) of the polypeptide or protein of interest and a standard polypeptide or protein are aligned using an art-acceptable protein alignment program, such as a BLAST™ pairwise alignment program or a well-known Lipman-Pearson protein alignment program, with the following alignment parameters. Databases (DBs) in which standard sequences are stored may be RefSeq non-redundant protein databases of NCBI. The parameters used for the sequence alignment may be as follows: Ktuple=2, Gap Penalty=4, and Gap length penalty=12. Here, a range included in a “corresponding” sequence may be within a range of E-value 0.00001 and H-value 0.001.

A polypeptide or protein having an amino acid residue corresponding to position L452 of the amino acid sequence of SEQ ID NO: 1 obtained according to the alignment conditions above may be also referred to as a “homolog of nitrous oxide reductase”. The nitrous oxide reductase may be, for example, derived from the genus Pseudomonas or the genus Paracoccus. The microorganism of the genus Pseudomonas may be P. stutzeri or P. aeruginosa. The microorganism of the genus Paracoccus may be P. versutus.

An “exogenous gene” as used herein refers to a gene that is not naturally present in a cell and is introduced into the cell from the outside of the cell. The introduced exogenous gene may be homologous or heterologous with respect to the host cell type into which the gene is introduced. The term “heterologous” means “not native” or “foreign.”

An aspect of the disclosure provides a recombinant microorganism including a foreign (i.e., exogenous) gene encoding a variant of a nitrous oxide reductase protein, wherein the variant includes an amino acid alteration of an amino acid residue corresponding to position L452 of the amino acid sequence of SEQ ID NO: 1 and has activity of an enzyme belonging to EC 1.7.2.4.

In the recombinant microorganism, nitrous oxide reductase may be an enzyme that catalyzes conversion of N₂O to N₂ using N₂O as a substrate. NosZ is a protein encoded by the nosZ gene, and is an enzyme that catalyzes the conversion of N₂O to N₂. That is, NosZ is nitrous oxide reductase. NosZ may be a homodimeric metalloprotein of 130 kDa that contains two copper centers, CuA and CuZ, in each monomer. The nitrous oxide reductase may have an activity of enzymes belonging to EC 1.7.2.4. The nitrous oxide reductase without the mutation described herein may be a natural variant or a variant that already includes a mutation in one or more amino acid residues. The mutation may be, for example, a substitution of an amino acid residue.

In the present specification, NosZ without the amino acid alteration may be a polypeptide having a sequence identity of greater than or equal to 75%, greater than or equal to 80%, greater than or equal to 85%, greater than or equal to 90%, greater than or equal to 95%, or greater than or equal to 98%, with respect to the amino acid sequence of SEQ ID NO: 1, 2, or 3. The nosZ gene may be a polynucleotide having a sequence identity of greater than or equal to 75%, greater than or equal to 80%, greater than or equal to 85%, greater than or equal to 90%, greater than or equal to 95%, or greater than or equal to 98%, with respect to the nucleotide sequence of SEQ ID NO: 49, 50, 51, 52, 53, 54, 55, 56, 57, 58, 59, 60, 61, 62, 63, 64, 65, 66, 67, 68, 69, 70, 71, 72, 73, 74, or 75.

The amino acid alteration may be a substitution of the amino acid residue corresponding to the L452 position with A, C, F, H, K, M, Q, or S. The amino acid alteration may be an L452A, L452C, L452F, L452H, L452K, L452M, L452Q, or L4525 substitution, or a combination thereof.

The variant may further include an amino acid alteration of an amino acid residue corresponding to position H78 of the amino acid sequence of SEQ ID NO: 1. The amino acid alteration may be a substitution of the amino acid residue corresponding to the H78 position with C, N, or Q. The amino acid alteration may be an H78C, H78N, or H78Q substitution, or a combination thereof. The amino acid alteration may be L4525 and H78C substitutions, L452A and H78N substitutions, L452K and H78N substitutions, L452A and H78C substitutions, L4520 and H78N substitutions, L4520 and H78C substitutions, L452M and H78N substitutions, L452M and H78C substitutions, or L452M and H78Q substitutions, or a combination thereof.

The microorganism may further include a genetic modification that increases expression of a nosR gene encoding NosR, a nosD gene encoding NosD, a nosF gene encoding NosF, a nosY gene encoding NosY, and an apbE gene encoding ApbE.

NosR protein may be encoded by the nosR gene, and may be a polytopic membrane protein serving as an electron donor for N₂O reduction. NosD protein may be encoded by the nosD gene, and may be essential for the formation of [4Cu:2S] copper center CuZ site in the NosD protein. In particular, NosD may supply sulfur (S) to NosZ. NosF and NosY, proteins encoded by the nosF gene and the nosY gene, respectively, may together form a complex, such as a tetramer, to serve as an ABC transporter. ApbE protein may be encoded by the apbEgene, and may be a flavinyltransferase that transfers flavin to NosR.

The nitrous oxide may be in the form of Fe(II)(L)-NO. The Fe(II)(L)-NO may refer to a complex formed by chelating L in the complex, which is a chelating agent, Fe 2+, and NO. L may be, for example, ethylenediamine, diethylenetriamine, triethylenetetramine, hexamethylenetetramine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylenetriaminepentaacetic acid (DTPA). Accordingly, the Fe(II)(L)-NO may be in a form such that a nitric oxide, such as N₂O, NO, N₂O₃, NO₂, N₂O₄, and N₂O₅, are modified to become soluble in an aqueous solution. The Fe(II)(L)-NO may be formed by contacting nitric oxide with a Fe(II)(L)-containing aqueous solution. The contacting may include mixing an aqueous medium with a liquid nitric oxide sample including the solubilized nitric oxide or contacting an aqueous medium with a sample including gaseous nitric oxide. However, in the reduction of the concentration of nitrous oxide in the sample using the recombinant microorganism, it is not necessarily limited to this or any such a particular mechanism.

The genetic modification may include a genetic modification for increasing the copy number of the nosZ gene, the copy number of the nosR gene, the copy number of the nosD gene, the copy number of the nosF gene, the copy number of the nosY gene, and the copy number of the apbE gene, or a combination thereof. The genetic modification may include introduction of the genes, for example, introduction of the genes through a vehicle such as a vector. The genes may be present intrachromosomally or extrachromosomally. The introduced genes may be present in a plurality of the copies of the genes. For example, the number of the introduced genes may each independently be 2 or more, 5 or more, 10 or more, 10 or more, 50 or more, 100 or more, or 1,000 or more.

NosZ may be a polypeptide having a sequence identity of at least 75% with respect to the amino acid sequence of SEQ ID NO: 1, 2, or 3. The nosZ gene may be a polynucleotide having a sequence identity of at least 75% with respect to the nucleotide sequence of SEQ ID NO: 49, 50, 51, 52, 53, 54, 55, 56, 57, 58, 59, 60, 61, 62, 63, 64, 65, 66, 67, 68, 69, 70, 71, 72, 73, 74, or 75.

NosR may be a polypeptide having a sequence identity of at least 75% with respect to the amino acid sequence of SEQ ID NO: 4, 7, or 10. The nosR gene may be a polynucleotide having a sequence identity of at least 75% with respect to the nucleotide sequence of SEQ ID NO: 5, 6, 8, 9, 11, or 12.

NosD may be a polypeptide having a sequence identity of at least 75% with respect to the amino acid sequence of SEQ ID NO: 13, 16, or 19. The nosD gene may be a polynucleotide having a sequence identity of at least 75% with respect to the nucleotide sequence of SEQ ID NO: 14, 15, 17, 18, 20, or 21.

NosF may be a polypeptide having a sequence identity of at least 75% with respect to the amino acid sequence of SEQ ID NO: 22, 25, or 28. The nosF gene may be a polynucleotide having a sequence identity of at least 75% with respect to the nucleotide sequence of SEQ ID NO: 23, 24, 26, 27, 29, or 30.

NosY may be a polypeptide having a sequence identity of at least 75% with respect to the amino acid sequence of SEQ ID NO: 31, 34, or 37. The nosY gene may be a polynucleotide having a sequence identity of at least 75% with respect to the nucleotide sequence of SEQ ID NO: 32, 33, 35, 36, 38, or 39.

ApbE may be a polypeptide having a sequence identity of at least 75% with respect to the amino acid sequence of SEQ ID NO: 40, 43, or 46. The apbE gene may be a polynucleotide having a sequence identity of at least 75% sequence identity with respect to the nucleotide sequence of SEQ ID NO: 41, 42, 44, 45, 47, or 48.

In the recombinant microorganism, each of the six genes, i.e., the nosZ gene, the nosR gene, the nosD gene, the nosF gene, the nosY gene, and the apbE gene, may be introduced into the recombinant microorganism through a vector. The vector may exist outside a chromosome of the recombinant microorganism (e.g., not incorporated/integrated in a chromosome of the recombinant microorganism.

In the recombinant microorganism, the nosZ gene and the nosR gene may be included in a first vector, and the nosD gene, the nosF gene, the nosY gene, and the apbE gene may be included in a second vector which may be different from the first vector in which the nosZ gene and the nosR gene are included.

In the recombinant microorganism, the nosZ gene and the nosR gene may be included in a first operon; the nosD gene and the nosF gene may be included in a second operon; the nosY gene and the apbE gene may be included in a third operon; and the nosD gene, the nosF gene, and the nosY gene may be included in a fourth of operon. Here, the operon in which the nosZ gene and the nosR gene are included, the operon in which the nosD gene and the nosF gene are included, the operon in which the nosY gene and the apbE gene are included, and the operon in which the nosD gene, the nosF gene, and the nosY gene are included may be included in vectors that may be different from each other.

The recombinant microorganism may belong to the genus Pseudomonas, the genus Paracoccus, or the genus Escherichia, or a combination thereof. The microorganism of the genus Pseudomonas may be P. stutzeri or P. aeruginosa, or a combination thereof. The microorganism of the genus Paracoccus may be P. versutus. The microorganism of the genus Escherichia may be E. coli.

The recombinant microorganism may reduce a concentration of nitrous oxide in a sample containing nitrous oxide. The reduction may include conversion of N₂O to N₂ or Fe(II)(L)-NO to N₂, by the nitrous oxide reductase. The sample may be in a liquid state or a gaseous state. The sample may be industrial wastewater or waste gas, or from another source. The sample may include any nitrogen oxide as nitrous oxide. The nitrogen oxide may include N₂O, NO, N₂O₃, NO₂, N₂O₄, N₂O₅, or a combination thereof.

Another aspect provides a composition for reducing a concentration of nitrous oxide in a sample, the composition including a recombinant microorganism including a foreign gene encoding a variant of a nitrous oxide reductase protein, wherein the variant includes an amino acid alteration of an amino acid residue corresponding to position L452 of the amino acid sequence of SEQ ID NO: 1 and has activity of an enzyme belonging to EC 1.7.2.4. Unless otherwise stated, the recombinant microorganism including a foreign gene encoding a variant of a nitrous oxide reductase protein or the variant of the nitrous oxide reductase protein is the same as described above.

The term “reduction” as used with respect to the composition refers to a reduction of a concentration of nitrous oxide present in a sample, and may include complete removal of nitrous oxide from the sample. The sample may be gas or liquid. The sample may not naturally include the recombinant microorganism. The composition may further include a substance that increases the solubility of nitrous oxide in a medium or a culture. The nitrous oxide may be in the form of Fe(II)(L)-NO.

The composition may be used for reducing a concentration of nitrous oxide in a sample by contacting it with the sample. The contacting may be performed in a liquid phase. The contacting may be achieved by, for example, contacting the sample with a culture of a microorganism cultured in a culture medium. The culturing may be achieved under conditions for proliferating microorganisms. The contacting may be performed in a closed (i.e., sealed) container. The contacting may be performed under anaerobic conditions. The contacting may include culturing or incubating the recombinant microorganism while contacting it with the sample including nitrous oxide. The contacting may include culturing the recombinant microorganism under conditions for proliferating the recombinant microorganism in a closed container. The culture medium may be a chemically defined culture medium. The expression “chemically defined medium” as used herein refers to a medium supplemented with a known chemical composition. Such a chemically defined culture medium may not contain a composite component such as serum or hydrolysate. A liquid medium may include an LB medium, an M9 medium, a phosphate buffer, a Tris buffer, and the like. The medium may contain Mg²⁺ ions in a concentration range of about 0.1 millimolar (mM) to about 7.5 mM, about 0.5 mM to about 7.5 mM, about 0.5 mM to about 5.0 mM, about 0.5 mM to about 2.5 mM, about 0.5 mM to about 1.5 mM, or about 1.0 mM to about 2.5 mM.

In the composition, the sample may be in a liquid state or a gaseous state. The sample may be industrial wastewater or waste gas.

Another aspect of the present disclosure provides a method of reducing a concentration of nitrous oxide in a sample that includes nitrous oxide, the method including contacting the sample with a recombinant microorganism, wherein the recombinant microorganism includes a foreign gene encoding a variant of a nitrous oxide reductase protein; or with a variant of a nitrous oxide reductase protein to reduce a concentration of nitrous in the sample, wherein the variant includes an amino acid alteration of an amino acid residue corresponding to position L452 of the amino acid sequence of SEQ ID NO: 1 and has activity of an enzyme belonging to EC 1.7.2.4. Unless otherwise stated, the recombinant microorganism including a foreign gene encoding a variant of a nitrous oxide reductase protein or the variant of the nitrous oxide reductase protein is the same as described above.

In the method, the contacting may be performed in a liquid phase. The contacting may be achieved by, for example, contacting the sample with a culture of the recombinant microorganism cultured in a medium. The culture may be achieved under conditions for proliferating microorganisms. The contacting may be performed in a closed container. The culture medium may be a chemically defined culture medium. Such a chemically defined culture medium may not contain a composite component such as serum or hydrolysate. A liquid medium may include, for example, an LB medium, an M9 medium, a phosphate buffer, a Tris buffer, and the like. The medium may contain Mg²⁺ ions in a concentration range of about 0.1 mM to about 7.5 mM, about 0.5 mM to about 7.5 mM, about 0.5 mM to about 5.0 mM, about 0.5 mM to about 2.5 mM, about 0.5 mM to about 1.5 mM, or about 1.0 mM to about 2.5 mM.

The contacting may be performed when the growth of the microorganism is at an exponential phase or a stationary phase. The culturing may be performed under anaerobic conditions. The contacting may be performed under conditions in which the recombinant microorganism is viable, for example in a closed container. The conditions in which the recombinant microorganism is viable may refer to conditions that allow the recombinant microorganism to proliferate or to remain in a proliferative state.

In the method, the sample may be in a liquid state or a gaseous state. The sample may be industrial wastewater or waste gas. The sample may include active contact as well as passive contact of the recombinant microorganism with the culture. The sample may be, for example, provided by sparging in a culture solution of the recombinant microorganism. That is, the sample may be blown through a medium or a culture solution. The sparging may be blowing from the bottom of a medium or a culture solution to the top. The sparging may be injecting droplets of the sample.

In the method, the contacting may be performed in a batch or in continuous manner. The contacting may include, for example, contacting the reduced, i.e. contacted sample obtained in the reducing step with a fresh recombinant microorganism. Such contacting with the fresh recombinant microorganism may be performed at least twice, for example, twice, 3 times, 5 times, or 10 times, or more. The contacting with the fresh recombinant microorganism may be continued or repeated for a period of time until the desired reduced concentration of nitrous oxide in the sample is achieved.

Another aspect provides a variant of a nitrous oxide reductase protein and a polynucleotide encoding the variant, wherein the variant includes an amino acid alteration of an amino acid residue corresponding to position L452 of the amino acid sequence of SEQ ID NO: 1 and has activity of an enzyme belonging to EC 1.7.2.4.

The amino acid alteration may be a substitution of the amino acid residue corresponding to the L452 position with A, C, F, H, K, M, Q, or S. The amino acid alteration may be an L452A, L452C, L452F, L452H, L452K, L452M, L452Q, or L4525 substitution.

The polynucleotide encoding the variant may be included in a vector. For use as the vector, any suitable material that is available to introduce a polynucleotide into a microorganism may be used. The vector may be a plasmid vector or a viral vector.

Another aspect provides a method of preparing a recombinant microorganism, the method including introducing a polynucleotide encoding a variant of a nitrous oxide reductase protein, wherein the variant includes an amino acid alteration of an amino acid residue corresponding to position L452 of the amino acid sequence of SEQ ID NO: 1 and has activity of an enzyme belonging to EC 1.7.2.4. The introducing of the polynucleotide may include introducing a vehicle including the polynucleotide into a microorganism.

The recombinant microorganism may belong to the genus Pseudomonas, the genus Paracoccus, or the genus Escherichia. The microorganism of the genus Pseudomonas may be P. stutzeri or P. aeruginosa, or a combination thereof. The microorganism of the genus Paracoccus may be P. versutus. The microorganism of the genus Escherichia may be E. coli.

The recombinant microorganisms according to an aspect may be used to remove nitrous oxide from a sample including nitrous oxide. The composition according to another aspect may be used to reduce the concentration of nitrous oxide in the sample.

The method of reducing the concentration of nitrous oxide in a sample including nitrous oxide according to another aspect may be utilized to effectively reduce the concentration of nitrous oxide in the sample.

The variant of the nitrous oxide reductase protein and the polynucleotide encoding the variant according to another aspect may be used to remove nitrous oxide from the sample or may be used in the method of preparing a microorganism that expresses the variant of the nitrous oxide reductase protein.

Hereinafter, the disclosure will be described in detail with reference to the Examples below. However, these Examples are provided for illustrative purposes only, and the scope of the disclosure is not limited thereto.

### Example 1: Recombinant E. coli Expressing Nitrous Oxide Reductase and a Variant Gene Thereof and the Removal of Nitrous Oxide from a Sample Using the Recombinant E. coli

In the present Example, recombinant E. coli expressing nitrous oxide reductase and a variant gene thereof was prepared, and an effect of removing nitrous oxide, i.e., N₂O or Fe(EDTA)-NO, from a sample was confirmed using the recombinant E. coli.

### (1) Preparation of Recombinant E. coli Expressing Nitrous Oxide Reductase and Variant Gene Thereof

From Pseudomonas stutzeri (ZoBell) which is a natural denitrification strain, a nosZ gene encoding nitrous oxide reductase (NosZ) that is a key enzyme, and auxiliary genes which are essential for the activity of the NosZ, such as nosR, nosD, nosF, nosy, and apbE genes, were extracted from a nos operon or gene cluster in the genome. The extracted genes were subjected to codon-optimization for E. coli, and then introduced thereto. Accordingly, by converting N₂O to N₂, a recombinant microorganism of the genus Escherichia having N₂ producibility, that is, a recombinant E. coli was prepared. To determine whether the recombinant E. coli had the N₂ producibility, the recombinant E. coli was cultured on a substrate labelled with a radioactive isotope ¹⁵ N, that is, in the presence of ¹⁵ N₂O and FeEDTA-¹⁵ NO. Then, the amount of ¹⁵ N₂ in the culture or the upper air layer of the culture supernatant was measured.

In addition, in the amino acid sequence of nitrous oxide reductase of SEQ ID NO: 1, the wild-type nosZgene was substituted with a gene encoding a variant of nitrous oxide reductase in which an amino acid residue at position L452 was substituted with one of 19 other natural amino acids. Then, it was tested in the same manner to determine whether recombinant E. coli including the variant gene had the N₂ producibility.

The function of each gene product (i.e., protein) was considered as follows. NosZ is a protein encoded by the nosZ gene, and is an enzyme that catalyzes the conversion of N₂O to N₂. That is, NosZ is nitrous oxide reductase. NosZ may be a homodimeric metalloprotein of 130 kDa that contains two copper centers, CuA and CuZ, in each monomer. NosR may be encoded by the nosR gene, and may be a polytopic membrane protein serving as an electron donor for N₂O reduction. NosD may be encoded by the nosD gene, and may be essential for the formation of [4Cu:2S] CuZ site. NosD may supply sulfur (S) to NosZ. NosF and NosY may be encoded by the nosF gene and the nosY gene, respectively, and may together form a complex, such as a tetramer, to serve as an ABC transporter. ApbE is a protein that may be encoded by the apbE gene, and may be a flavinyltransferase that transfers flavin to NosR.

### 2. Preparation of Vector and Preparation of Recombinant E. coli Transformed with Prepared Vector

### (1) Preparation of Vector

As expression vectors, pETDuet™-1 and pACYCDuet™1 vectors were used. The pETDuet™-1 vector was designed to operably link a lac operator to a T7 promoter, and contained an ampicillin resistance gene, Amp^{R}, as a selective marker. The pACYCDuet™-1 vector was designed to operably link a lac operator to a T7 promoter, and contained a chloramphenicol resistance gene, Cm^{R}, as a selective marker.

NosZ may be a polypeptide having a sequence identity of at least 75% with respect to the amino acid sequence of SEQ ID NO: 1, 2, or 3. The nosZ gene may be a polynucleotide having a sequence identity of at least 75% with respect to the nucleotide sequence of SEQ ID NO: 49, 50, 51, 52, 53, 54, 55, 56, 57, 58, 59, 60, 61, 62, 63, 64, 65, 66, 67, 68, 69, 70, 71, 72, 73, 74, or 75.

NosR may be a polypeptide having a sequence identity of at least 75% with respect to the amino acid sequence of SEQ ID NO: 4, 7, or 10. The nosR gene may be a polynucleotide having a sequence identity of at least 75% with respect to the nucleotide sequence of SEQ ID NO: 5, 6, 8, 9, 11, or 12.

NosD may be a polypeptide having a sequence identity of at least 75% with respect to the amino acid sequence of SEQ ID NO: 13, 16, or 19. The nosD gene may be a polynucleotide having a sequence identity of at least 75% with respect to the nucleotide sequence of SEQ ID NO: 14, 15, 17, 18, 20, or 21.

NosF may be a polypeptide having a sequence identity of at least 75% with respect to the amino acid sequence of SEQ ID NO: 22, 25, or 28. The nosF gene may be a polynucleotide having a sequence identity of at least 75% with respect to the nucleotide sequence of SEQ ID NO: 23, 24, 26, 27, 29, or 30.

NosY may be a polypeptide having a sequence identity of at least 75% with respect to the amino acid sequence of SEQ ID NO: 31, 34, or 37. The nosY gene may be a polynucleotide having a sequence identity of at least 75% with respect to the nucleotide sequence of SEQ ID NO: 32, 33, 35, 36, 38, or 39.

ApbE may be a polypeptide having a sequence identity of at least 75% with respect to the amino acid sequence of SEQ ID NO: 40, 43, or 46. The apbE gene may be a polynucleotide having a sequence identity of at least 75% sequence identity with respect to the nucleotide sequence of SEQ ID NO: 41, 42, 44, 45, 47, or 48.

The origin and characteristics of the above-described proteins and the nucleotides encoding the same are set forth in the Sequence Listing. Among the genes used in the expression vector in E. coli in the present Example, nucleotide sequences of the native gene were optimized in consideration of the codon frequency used in E. coli, and information thereof is described in the sequence list.

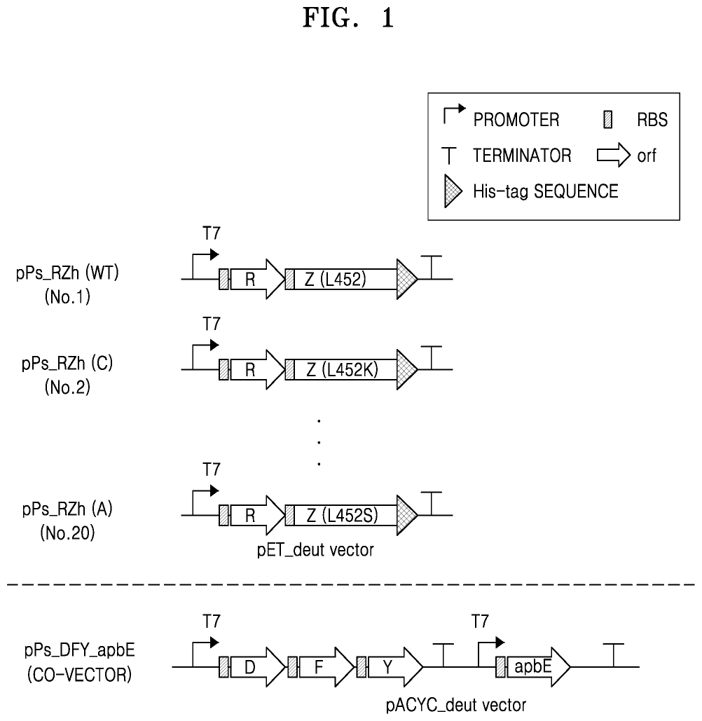

FIG. 1 is a diagram of a vector introduced into E. coll. In FIG. 1, No. 1 and Nos. 2 to 20 each represent a pET-deut vector including the nosR gene and the nosZ gene or a variant of the nosZ gene. Nos. 2 to 20 each represent a vector including a variant gene substituted with one of 19 amino acids instead of the natural amino acid L at L452. Then, one of these vectors and a pPs_DFY_apbE vector including the nosD gene, the nosF gene, the nosY gene, and the apbE gene were co-introduced into E. coll. The pPs_DFY_apbE vector is a vector prepared based on the pACYC-deut vector.

In each of the vectors numbered 1 to 20, the nosR gene and the nosZ gene or the variant of the nosZ gene were commonly operably linked to the T7 promoter in the pET-deut vector, and a ribosome binding site (RBS) included an E. coli RBS sequence, AAGGAG. Here, the nosZ gene included a sequence encoding a his-tag. In the pPs_DFY_apbE vector, the nosD gene, the nosF gene, and the nosY gene derived from P. stutzeri were operably linked to the T7 promoter in a pACYC-duet vector, and the apbE gene was operably linked to a different T7 promoter. Here, an RBS of each gene included an E. coli RBS sequence, AAGGAG.

### (2) Preparation of Recombinant E. coli Having N₂ Producibility and Confirmation of Activity Thereof

Two vectors, i.e., one of the vectors numbered to 1 to 20 prepared in Section (1) and the pPs_DFY_apbE vector, were introduced into E. coli C43 (DE3) by transformation to prepare recombinant E. coll. The transformation was performed by an electroporation method.

(2.1) NosZ Maturation Step Culture

The recombinant E. coli was cultured in 2xYT medium containing 50 micrograms per milliliter (ug/mL) of riboflavin and 0.25 mM CuCl₂ in an Erlenmeyer flask (supplemented with 16 g of tryptone, 10 g of yeast extract, and 5 g of NaCl and having a final pH of 7.0 (at 25° C.) until OD₆₀₀ reached 0.6 while stirring at 230 revolutions per minute (rpm) at 37° C. Then, 1 mM IPTG was added thereto and cultured overnight to induce gene expression while stirring at 140 rpm at 30° C. Next, cells were harvested and used for the subsequent N₂ production reaction.

(2.2) N₂ Production Culture from Fe(II)EDTA-¹⁵ NO

The recombinant E. coli cells obtained in Section (2.1) were added to M9 medium (pH 7.0) containing 5 gram per liter (g/L) of glucose and 5 mM Fe(II)EDTA-¹⁵ NO until OD₆₀₀ reached 1, thereby obtaining a reaction mixture.

30 mL of the reaction mixture was added to a 60 mL serum bottle, and cultured while stirring at 30° C. at 140 rpm. The serum bottle was kept in an anaerobic chamber to be maintained under anaerobic conditions. A control group was prepared in the same manner, except that a recombinant E. coli including a vector with the wild-type nosZgene was used.

Next, the amount of ¹⁵ N₂ produced was analyzed with GC-MS by sampling the gas in the headspace of the reaction serum bottle.

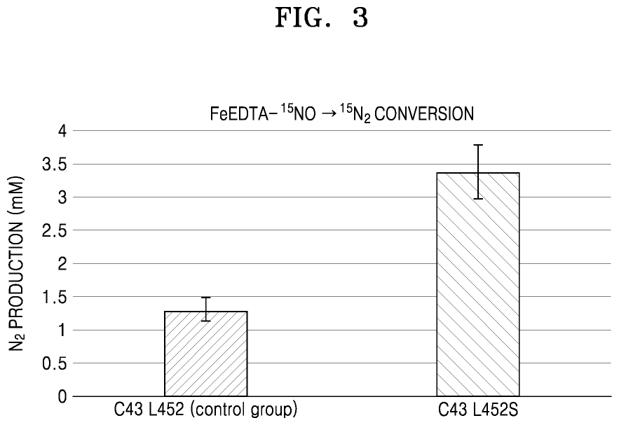

The results are shown in FIG. 3. FIG. 3 is a bar graph showing results of converting NO in the form of Fe(II)EDTA-¹⁵ NO to N₂ using the recombinant E. coli including the wild-type nosZ gene (control, left bar) and the variant of the nosZ gene (right bar).

As shown in FIG. 3, when the recombinant E. coli including a variant gene, i.e., L4525, was used, the N₂ production was significantly increased after 22 hours, compared to the E. coli BL31 (C43) with the vector including the wild-type nosZ gene. The E. coli BL31(C43) with the vector including the wild-type nosZ gene and the recombinant E. coli with the L4525 variant gene produced 1.3 mM N₂ and 3.4 mM N₂, respectively.

### Example 2: Evaluation of In Vitro Activity of Recombinant Nitrous Reductase (NosZ) Variant

Recombinant E. coli C43 (DE3) including two vectors, i.e., one of the vectors numbered 1 to 20 and the pPs_DFY_apbE vector, prepared in Example 1 was cultured, and a cell lysate was obtained from the culture and purified. Then, the activity of purified nitrous reductase (NosZ) was evaluated in vitro.

(1) NosZ Maturation Step Culture

The recombinant E. coli was cultured in 2xYT medium containing 50 ug/mL of riboflavin and 0.25 mM CuCl₂ in an Erlenmeyer flask until OD₆₀₀ reached a value of 0.6 at 37° C. Then, 1 mM IPTG was added thereto and cultured overnight to induce gene expression while stirring at 140 rpm at 30° C. Next, the recombinant E. coli was disrupted by sonication in a lysis buffer (containing 50 mM NaH₂PO₄, 300 mM NaCl, and 10 mM imidazole at pH 8.0). As a result, a cell lysate was obtained, and a Ni-NTA affinity column was used for the cell lysate to purify NosZ from the cell lysate by a general method using the following two buffers: Ni-NTA washing buffer (containing 50 mM NaH₂PO₄, 300 mM NaCl, and 20 mM imidazole at pH 8.0); and Ni-NTA elution buffer (containing 50 mM NaH₂PO₄ 300 mM NaCl, and 250 mM imidazole at pH 8.0). Afterwards, the purified NosZ was brought into contact with N₂O for use in the N₂ production reaction.

(2) N₂ Production from ¹⁵ N₂O by Purified NosZ

An aqueous reaction solution was prepared by adding 0.2 mg/ml of the purified NosZ obtained in Section (1), 2.0 mM benzyl viologen, 1.0 mM sodium dithionite, and 1.25 mM ¹⁵ N₂O (g) to water (pH 7.0). 30 mL of the aqueous reaction solution was added to a 60 mL serum bottle, and cultured while stirring at 30° C. at 140 rpm. A stopper was used to seal the bottle under anaerobic conditions. A control group was prepared in the same manner, except that an aqueous solution containing bovine serum albumin (BSA) was used.

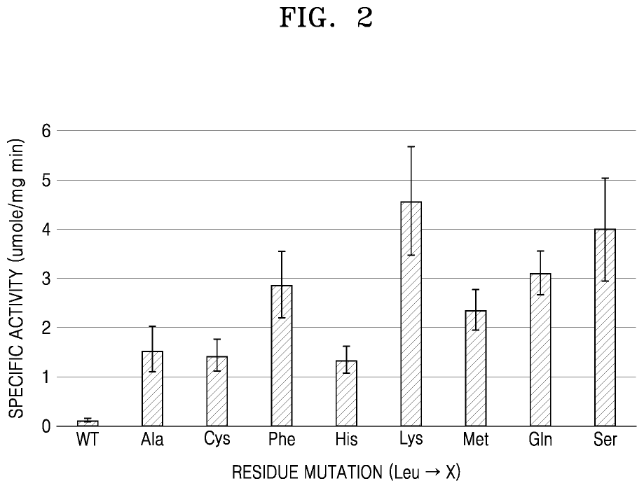

Next, the amount of ¹⁵ N₂ produced was analyzed with GC-MS by sampling the gas in the headspace of the reaction serum bottle. The results are shown in Table 1 (where umole/mg min is micromole per milligram per minute) and FIG. 2.

TABLE 1

| Name of NosZ | Activity of converting N₂O → N₂ | Multiplication Δ |
|---|---|---|
| variant | (umole/mg min) | (%) |
| Leu (wild-type) | 0.13 | 100 |
| L452A | 1.56 | 1180.2 |
| L452C | 1.44 | 1091.4 |
| L452F | 2.88 | 2181.1 |
| L452H | 1.33 | 1006.4 |
| L452K | 4.59 | 3471.7 |
| L452M | 2.36 | 1789.4 |
| L452Q | 3.12 | 2358.9 |
| L452S | 3.99 | 3019.8 |

As shown in Table 1 and FIG. 2, compared to wild type (WT) L452, the L452A, L452C, L452F, L452H, L452K, L452M, L4520, or L452S variant had excellent in vitro activity of converting N₂O to N₂.

In addition, by introducing an additional mutation to the position H78 in addition to the amino acid mutation at the position L452, a double variant with two mutations was prepared, and the activity of the double variant NosZ variant was then measured. The preparation, production, and enzyme activity measurement of the double variant were performed in the same manner as described above. The results are shown in Table 2.

TABLE 2

| Name of NosZ | Activity of converting N₂O → N₂ | Multiplication Δ |
|---|---|---|
| variant | (umole/mg min) | (%) |
| WT | 0.158 | 100 |
| L452S/H78C | 0.7156 | 452.9 |
| L452A/H78N | 0.7451 | 471.6 |
| L452K/H78N | 0.755 | 477.8 |
| L452A/H78C | 1.2407 | 785.3 |
| L452Q/H78N | 1.7589 | 1113.2 |
| L452Q/H78C | 2.2204 | 1405.3 |
| L452M/H78N | 3.35 | 2120.3 |
| L452M/H78C | 3.42 | 2164.6 |
| L452M/H78Q | 16.351 | 10348.7 |

As shown in Table 2, compared to wild type (WT)L452, the double variant L452S/H78C, L452A/H78N, L452K/H78N, L452A/H78C, L452Q/H78N, L452Q/H78C, L452M/H78N, L452M/H78C, or L452M/H78Q had excellent in vitro activity of converting N₂O to N₂.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A recombinant microorganism comprising a foreign gene encoding a variant of a nitrous oxide reductase protein, wherein the variant comprises an amino acid alteration of an amino acid residue corresponding to position L452 of the amino acid sequence of SEQ ID NO: 1 and has activity of an enzyme belonging to EC 1.7.2.4.

2. The microorganism of claim 1, wherein the amino acid alteration is a substitution of the amino acid residue corresponding to position L452 with A, C, F, H, K, M, Q, or S.

3. The microorganism of claim 1, wherein the amino acid alteration is an L452A, L452C, L452F, L452H, L452K, L452M, L452Q, or L4525 substitution.

4. The microorganism of claim 1, wherein the variant further comprises an amino acid alteration of an amino acid residue corresponding to position H78 of the amino acid sequence of SEQ ID NO: 1.

5. The microorganism of claim 1, further comprising a genetic modification that increases expression of a nosR gene encoding NosR, a nosD gene encoding NosD, a nosF gene encoding NosF, a nosY gene encoding NosY, and an apbE gene encoding ApbE.

6. The microorganism of claim 1, wherein the microorganism belongs to the genus Pseudomonas, the genus Paracoccus, or the genus Escherichia, or a combination thereof.

7. A composition for reducing a concentration of nitrous oxide in a sample, the composition comprising a recombinant microorganism comprising a foreign gene encoding a variant of a nitrous oxide reductase protein, wherein the variant has an amino acid alteration of an amino acid residue corresponding to position L452 of the amino acid sequence of SEQ ID NO: 1 and has activity of an enzyme belonging to EC 1.7.2.4.

8. The composition of claim 7, wherein the amino acid alteration is a substitution of the amino acid residue corresponding to position L452 with A, C, F, H, K, M, Q, or S.

9. The composition of claim 7, wherein the amino acid alteration is an L452A, L452C, L452F, L452H, L452K, L452M, L452Q, or L4525 substitution.

10. The composition of claim 7, wherein the variant further comprises an amino acid alteration of an amino acid residue corresponding to position H78 of the amino acid sequence of SEQ ID NO: 1.

11. The composition of claim 7, wherein the microorganism further comprises a genetic modification that increases expression of a nosR gene encoding NosR, a nosD gene encoding NosD, a nosY gene encoding NosF, a nosY gene encoding NosY, and an apbE gene encoding ApbE.

12. The composition of claim 7, wherein the microorganism belongs to the genus Pseudomonas, the genus Paracoccus, or the genus Escherichia, or a combination thereof.

13. A method of reducing a concentration of nitrous oxide in a sample, the method comprising contacting a sample comprising nitrous oxide, with a recombinant microorganism comprising a foreign gene encoding a variant of a nitrous oxide reductase protein or with a variant of a nitrous oxide reductase protein, wherein the variant comprises an amino acid alteration of an amino acid residue corresponding to position L452 of the amino acid sequence of SEQ ID NO: 1 and has activity of an enzyme belonging to EC 1.7.2.4.

14. The method of claim 13, wherein the amino acid alteration is a substitution of the amino acid residue corresponding to position L452 with A, C, F, H, K, M, Q, or S.

15. The method of claim 13, wherein the variant further comprises an amino acid alteration of an amino acid residue corresponding to position H78 of the amino acid sequence of SEQ ID NO: 1.

16. The method of claim 13, wherein the microorganism further comprises a genetic modification that increases expression of a nosR gene encoding NosR, a nosD gene encoding NosD, a nosY gene encoding NosF, a nosY gene encoding NosY, and an apbE gene encoding ApbE.

17. The method of claim 13, wherein the microorganism belongs to the genus Pseudomonas, the genus Paracoccus, or the genus Escherichia, or a combination thereof.

18. The method of claim 13, wherein the contacting comprises culturing or incubating the recombinant microorganism while contacting the recombinant microorganism with the sample comprising nitrous oxide.

19. A variant of nitrous oxide reductase, comprising an amino acid alteration of an amino acid residue corresponding to position L452 of the amino acid sequence of SEQ ID NO: 1, the variant having activity of an enzyme belonging to EC 1.7.2.4, wherein the amino acid alteration is a substitution of the amino acid residue corresponding to position L452 with A, C, F, H, K, M, Q, or S.

20. A polynucleotide encoding a variant of nitrous oxide reductase, wherein the variant comprises an amino acid alteration of an amino acid residue corresponding to position L452 of the amino acid sequence of SEQ ID NO: 1 and has activity of an enzyme belonging to EC 1.7.2.4, and wherein the amino acid alteration is a substitution of the amino acid residue corresponding to position L452 with A, C, F, H, K, M, Q, or S.
